(19) United States
(12) Patent Application Publication
(10) Pub. No.: US 2025/0360172 A1
(43) Pub. Date: Nov. 27, 2025

(54) MICROGEL-ENCAPSULATED IPSC-DERIVED NOTOCHORDAL CELLS FOR TREATMENT OF INTERVERTEBRAL DISC DEGENERATION AND DISCOGENIC PAIN

(71) Applicant: Cedars-Sinai Medical Center, Los Angeles, CA (US)

(72) Inventors: Dmitriy Sheyn, Los Angeles, CA (US); Wensen Jiang, Culver City, CA (US); Juliane Glaeser, Los Angeles, CA (US); Wafa Tawackoli, Beverly Hills, CA (US)

(73) Assignee: Cedars-Sinai Medical Center, Los Angeles, CA (US)

(21) Appl. No.: 18/867,135

(22) PCT Filed: May 25, 2023
(86) PCT No.: PCT/US2023/023583

§ 371 Date: Nov. 19, 2024

Related U.S. Application Data
(60) Provisional application No. 63/345,841, filed on May 25, 2022.

(51) Int. Cl. A61K 35/545; A61K 9/00; A61K 9/10; A61K 9/50; A61P 19/02

(52) U.S. Cl. CPC A61K 35/545; A61K 9/0019; A61K 9/10; A61K 9/5031; A61K 9/5052; A61P 19/02

(57) ABSTRACT

Injectable compositions and methods of preparation, as well as therapeutic uses, of induced pluripotent stem cell (iPSC)-derived notochordal cell (iNC)-loaded microgels are provided. Microfluidic on-chip platform can be utilized to prepare microgels (or microgel particles/spheres) formed from block copolymers that exhibit reverse thermal gelation, so as to encapsulate iNCs. Also provided are preconditioned iNC-loaded microgels and iNCs in bulk hydrogel. Cell purity, identity, viability, sterility, and the stability of microencapsulated iNCs have been evaluated. Safety and efficacy of the compositions as therapeutic candidates has been tested via intradiscal injection in animal models of intervertebral disc (IVD) degeneration and discogenic low back pain. Biobehavioral testing, MRI, and immunohistochemical analyses were utilized to evaluate the regenerative potential and reproducibility of the compositions as therapeutic candidate. Single cell RNA sequencing of the treated IVDs may also reveal mechanism of action of the compositions.

### CROSS-REFERENCE TO RELATED APPLICATIONS

This application includes a claim of priority under 35 U.S.C. § 119 (e) to U.S. provisional patent application No. 63/345,841, filed May 25, 2022, the entirety of which is hereby incorporated by reference.

### STATEMENT REGARDING FEDERALLY SPONSORED RESEARCH OR DEVELOPMENT

This invention was made with Government support under grant no. NS126032 awarded by the National Institutes of Health. The Government has certain rights in the invention.

### FIELD OF INVENTION

This invention relates to iPSC-derived notochordal cells delivered in micron-sized hydrogels for injection and treatment of disease and conditions in the spine.

### BACKGROUND

Low back pain (LBP) is a leading cause of disability and morbidity in the adult population, affecting approximately 80% of adults within their lifetime. Up to 40% of all LBP is attributed to discogenic pain from intervertebral disc (IVD) degeneration. While people of different races, ethnicity and gender suffer from chronic back pain, this disease has been shown to affect often people from underserved communities. Despite decades of research, robust therapies targeting underlying causes rather than symptoms of IVD degeneration are still in the earliest stages of development. Conservative treatments of IVD degeneration include oral analgesics and muscle relaxants, or surgical spinal decompression procedures, which aim to alleviate symptoms rather than target the underlying disease. Broad-spectrum analgesics that are often prescribed to treat chronic LBP include opioids. A 2007 review of 11 studies identified opioids to be prescribed at rates as high as 66% for chronic LBP. Further rates of substance abuse disorders among patients prescribed with opioids for LBP ranges from 5% to 25%. Moreover, increased opioid prescribing contributes to the dramatic increase in fatal drug overdoses. Between 1999 and 2010, opioid-related deaths increased 5-fold for women and 3.6-fold for men.

The IVD consists of an outer anulus fibrosus (AF), which is rich in collagens that account for its tensile strength, and an inner nucleus pulposus (NP), which contains large proteoglycans (PGs) that retain water for resisting loading by compression. The NP is formed from the notochord as it segments during fetal development. At birth, the NP is populated by morphologically distinct, large vacuolated notochordal cells (NCs). In some vertebrates these NCs persist throughout adulthood, whereas in others, including humans, the NCs gradually disappear during maturation, and eventually become undetectable and replaced by smaller NP cells. Animals that keep their NCs, such as rabbits and rodents, show no signs of degeneration and maintain a more hydrated, proteoglycan-rich matrix compared to adult human NP. IVD degeneration is known to affect the NP, the central part of the IVD. IVD degeneration is characterized by breakage of the NP matrix due to elevated expression of inflammatory factors (e.g., cytokines) and metalloproteinases (or their activities) and altered (decreased) matrix production. In addition, cell apoptosis and formation of cell clusters during the degeneration, due to accelerated cell replication, can lead to cell senescence. The IVD has a limited capability for intrinsic regeneration, probably due to lack of progenitors and vascularity in the NP. Autologous NP cells have been shown to halt degeneration in an animal model of IVD degeneration (Hohaus, C. et al., Eur Spine J 17 Suppl 4, 492-503 (2008)). A clinical trial has demonstrated pain relief and disc hydration upon NP cell injection into degenerated IVDs (Meisel H J, et al. Biomol Eng. February 2007; 24(1): 5-21). However, harvesting NP cells yields in limited quantities and requires an invasive procedure, which itself has been shown to initiate degeneration. Also, using NP cells sourced from degenerated IVDs may be inadequate for regeneration due to a reduced expression of matrix proteins, increased expression of degradation enzymes, and a high cell senescence. In line with this, a previous study demonstrated an impaired differentiation capacity and matrix secretion of porcine degenerated NP-derived cells (Mizrahi, O. et al., Spine J 13, 803-14 (2013)). Other studies have shown a moderate therapeutic effect of mesenchymal stem cell (MSC) injection to the IVD (Orozco, L. et al., Transplantation 92, 822-8 (2011); Mwale, F. et al., Tissue Eng Part A 20, 2942-9 (2014)). However, these cells have been found to induce mineralization and ossification of the injured IVD, which impairs its function as load-bearing unit of the spine.

Current clinical treatments for discogenic pain focus on alleviating symptoms rather than targeting the mechanism of the underlying disease. Thus, there is an urgent need for a disease modifying therapy that directly targets the pathogenesis of IVD degeneration. Notochordal cells (NCs), the precursors to the cells that populate the NP of a mature IVD, are essential for IVD homeostasis. During development, notochordal cells (NCs) give rise to mature NP cells; in humans, the NC population is reported to vanish at the age of 10. Induced pluripotent stem cells can be differentiated to notochordal cells (iNC) using protocols that mimic the differentiation process that occurs during embryogenesis (Sheyn, D. et al. Theranostics 9, 7506-7524 (2019)).

Despite the regenerative potential of iNCs, challenges impede the translation of the iNC technology. The limited cell survival and poor engraftment caused by the harsh microenvironment in the degenerated IVD may hinder the iNC cell therapy success. One study demonstrated a site retention rate of 5% of injected cells in the IVD (Amer, M. H. et al., NPJ Regenerative medicine 2, 1-13 (2017)). Factors affecting the cell survival in the degenerated IVD include catabolic and inflammatory proteins, and a low pH. Yet, the viability and activity of iNCs delivered in a bulk gel are suboptimal, and host integration of delivered iNCs may be hindered by some bulk hydrogel, thereby limiting the therapeutic potential of these cells.

Therefore, it is an objective of the present invention to provide a composition for delivery of therapeutic cells with an improved cell viability, activity, and host integration at sites of IVD degeneration.

It is another objective of the present invention to provide methods for treating IVD degeneration and/or managing discogenic pain, especially without the use of opioids.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiment provide injectable compositions, which include or consist of a dispersion comprising microgel particles and human induced pluripotent stem cell (iPSC)-derived notochordal cells (iNCs), wherein the iNCs are encapsulated in the microgel particles, and the size of the microgel particles is between 30 μm and 1000 μm.

Preferably the iNCs are cultured with the microgel particles for a period of time, in some aspects under hypoxic conditions, so that the iNCs secrete extracellular matrix proteins in the microgel particles. In some embodiments, the iNCs secrete collagen type II, and the microgel particles encapsulating the iNCs are deposited with the collagen type II. In some embodiments, the injectable compositions are or have been cultured in a nucleus pulposus (NP)-specific medium in a hypoxic condition for a period of time selected for the iNCs to secrete an extracellular matrix protein comprising collagen type II. In some embodiments, culturing the microgel particles which encapsulate the iNCs in a hypoxic condition for a period of time selected for inducing secretion of an extracellular matrix protein comprising collagen type II by the iNCs and/or for maintaining of at least 50% activity of the iNCs in the microgel particles compared to before encapsulation.

In various embodiments, the microgel particles each includes or is made up of a cross-linked polymeric network (e.g., in aqueous environment), and the polymeric network contain therein or is consisted of: a plurality of first polymeric segments derived from a polyoxyalkylene, and a plurality of second polymeric segments derived from a bioadhesive polypeptide or polysaccharide, wherein the first polymeric segments and the second polymeric segments are bonded together to form a polymeric network. In various aspects, polymeric segments derived from a compound means the polymeric segment being the compound in a bonded state or have a valency for bonding (with another segment).

In some embodiments, the polymeric network includes one or more linking groups connecting the first polymeric segments to the second polymeric segments, optionally the linking groups comprising an ester group or being derived from an acrylate.

In some embodiments, the bioadhesive polypeptide or polysaccharide comprises fibrinogen, laminin, or hyaluronic acid. In some embodiment, the bioadhesive polypeptide or polysaccharide is fibrinogen, fibrin, or a fragment thereof. In some embodiment, the bioadhesive polypeptide or polysaccharide is fibrinogen. In some embodiment, the bioadhesive polypeptide or polysaccharide is laminin. In some embodiment, the bioadhesive polypeptide or polysaccharide is hyaluronic acid.

In some embodiments, the bioadhesive polypeptide or polysaccharide presents or is coupled with a thiol group, and the polyoxyalkylene is coupled with an acrylate group; so that the polymer network is formed with a plurality of the polypeptide/polysaccharide segment derived from the thiol-modified polypeptide/polysaccharide and a plurality of the polyoxyalkylene segment derived from the acrylate-modified polyoxyalkylene.

In various embodiments, the polyoxyalkylene comprises at least one block derived from propylene oxide monomers. In various embodiments, the polyoxyalkylene comprises at least one block derived from propylene oxide monomers and at least one block derived from ethylene oxide monomers. In various embodiments, the polyoxyalkylene is an ABA triblock copolymer, wherein the A blocks are derived from the ethylene oxide monomers and the B block is derived from the propylene oxide monomers. In some embodiments, the polyoxyalkylene is or includes a poloxamer. In some embodiments, the polyoxyalkylene is or includes a poloxamine.

In various embodiment, the iNCs are prepared by a process including the steps of: culturing human iPSCs in the presence of a glycogen synthase kinase 3 (GSK3) inhibitor (GSK3i) to form primitive streak (PS) cells; transfecting the PS cells with a vector encoding Brachyury to overexpress Brachyury; expressing Brachyury in the PS cells, wherein expression of Brachyury by the vector encoding Brachyury in the PS cells induces formation of human iNCs, and the human iNCs express Brachyury, Keratin 18, and Keratin 19.

In various embodiments, the microgel particles are between 50 μm and 250 μm in size, and the iNCs are encapsulated in the microgel particles at a number ratio of iNC-to-microgel particle being between 1:1 and 80:1.

Methods are also provided for treating a subject with intervertebral disc degeneration and/or discogenic low back pain. Methods are also provided for modulating the intervertebral disc degeneration in the subject. In various implementations, the methods of treatment include injecting an effective amount of an injectable composition disclosed herein into a nucleus pulposus, a vertebral disc, an invertebral disc, or clefts of a nucleus pulposus of an intervertebral disc of the subject.

In some embodiments, the injectable composition is intradiscally injected to the nucleus pulposus of the subject. In some embodiments, at least 1×10⁶, 2×10⁶, or 3×10⁶ human iNCs are administered to the subject, and wherein the microgel particles each comprises a cross-linked polymeric network comprising a plurality of poloxamer segments and a plurality of fibrinogen segments, wherein the poloxamer segments and the fibrinogen segments are bonded together via linking groups to form the polymeric network.

In some embodiments, treating the subject and/or modulating the intervertebral disc degeneration results in an increase in disc height and/or an increase in cold hypersensitivity of the subject.

Additional embodiments provide methods for preparing the injectable composition disclosed herein. In some embodiments, a method for the preparation includes the steps of: mixing an aqueous solution comprising a precursor polymer to forming the microgel particles with the iNCs to form a precursor-cell mixture; subjecting the precursor-cell mixture to microinjection or micronization into an oil phase, wherein the precursor-cell mixture is microinjected or micronized to form a dispersion of microparticles in the oil phase; curing the microparticles in response to a stimulus selected for inducing gelation of the microparticles and purifying the microparticles to remove residue from the oil phase, thereby forming a dispersion of microgel particles which encapsulate the iNCs. In some embodiments, the precursor polymer includes or contains therein a first polymeric segment derived from polyoxyalkylene and a second polymeric segment derived from a bioadhesive polypeptide or polysaccharide, wherein the first polymeric segment and the second polymeric segment are bonded together. In some embodiments, the stimulus is an increase in temperature or an exposure to ultraviolet or visible light.

In some embodiments, the aqueous solution viscosifies in response to the stimulus (e.g., increase in temperature), and the microparticles formed from the precursor-cell mixture is thermal-cured to form the dispersion of microgel particles.

In some embodiments, the first polymeric segment and/or the second polymeric segment is modified with a photo-reactive chemical group, such that the aqueous precursor solution becomes reactive in response to the stimulus (e.g., the exposure to ultraviolet or visible light), and the microparticles formed from the precursor-cell mixture is photo-cured to form the dispersion of microgel particles.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A depicts an overview of a study of iNCs encapsulated in microgels, microtissues or delivered in bulk hydrogel as therapeutic candidates for injectable discogenic LBP treatment.

FIG. 1B depicts an experimental design of preparation of iNCs encapsulated in microgels, microtissues or delivered in bulk hydrogel.

FIG. 2A depicts an experimental design to determine the survival of human iNCs in SD rat IVDs.

FIG. 2B depicts an experimental design to determine therapeutic efficacy of different delivery systems in mitigating IVD degeneration and LBP biobehavioral outcomes.

FIG. 3A-3G depicts preparation of “type 1” microgels from a microfluidics system and the use as intradiscal cell delivery vehicle. Type 1 microgel was prepared using a thermal reversal method; with a diameter of 100 μm-200 μm. (3A) Fibrinogen-F127 (FF) microgels stained with Trypan blue gelatinized in 37° C. media. (3B) FF Microgels loaded with nucleus pulposus-derived cells (NPCs). (3C) FF gel is liquid at 4° C. (panel C1) and gelatinized at 37° C. (panel C2). (3D) Rheological property of FF microgel: storage modulus, G′, and loss modulus, G″, changed with temperature ramp. (3E) NPC maintained high viability in microgels 7 days after microencapsulation at different cell densities. (3F) NPC stained with Dil cell-labeling dye (green) prior to encapsulation were imaged in 37° C. media after 21 days of culture. Scale bar=1,000 μm. (3G) Fluorescent imaging four days after intradiscal injection of a green fluorescent protein (GFP) plasmid with nanoparticle carrier into rat IVD.

FIG. 3H depicts, on the left, that FF microgel passing through different needles (25G, 27G and 30G) demonstrated a reduced cell viability when 30G was used, and on the right, that cells maintained high viability in microgel 7 days after microencapsulation at different cell densities.

FIG. 3I depicts an FF microgel for intradiscal cell delivery vehicle. Microfluidic device was used to fabricate microgels (shown in upper panel). Water colored with Rhodamine was firstly tested in the device (bottom left). Homogenous water microgels were imaged under fluorescence microscope. Cell-laden microgels made from the microfluidic device were passing through the spiral structure of the device. Homogenous cell-laden microgels of about 200 μm in diameter can be seen in the bottom right panel of the figure.

FIG. 4 depicts characterizations of poly(lactic-co-glycolic acid) (PLGA) microparticles. (Upper row) Microfluidic device capable of generating PLGA MPs: a continuous phase of 1 w/v % polyvinyl alcohol (PVA) in water, and a dispersed phase of PLGA and calcium peroxide (CPO, CaO₂) in dichloromethane (DCM), wherein CaO₂ can act as an oxygen-generating system to enhance tissue oxygenation in the PLGA microparticles (e.g., 3% CPO in the microparticles). (Lower row) Scanning electron microscopy image of MPs with CPO (scale bars=100 μm).

FIG. 5 depicts schematics for preparation of a fibrinogen-F127 adduct and thermal gelation, as well as UV-induced crosslinking gelation, of fibrinogen-F127 hydrogel. (Prior Art, Shachaf et al., Biomaterials, Volume 31, Issue 10, April 2010, Pages 2836-2847).

FIG. 6 depicts the differentiation of iPSCs to iNC and their testing in a large animal model. (panel A) Stepwise iNC differentiation. (panel B) porcine IVD degeneration induction. (panel C) iNC intradiscal injection in vivo. (panel D) MRI follow-up during the experiment. (panel E) Quantitation of qCEST imaging indicating pH levels in vivo. (panel F) Histology & immunohistochemistry of porcine IVDs at week 8. (Prior Art, modified from figures in Sheyn, D. et al. Theranostics 9, 7506-7524 (2019)).

FIG. 7 depicts that μMRI shows no IVD degeneration was induced by saline or stressed(s) NPC injections. (Panel A) T1 and T2 images. (Panel B) Quantification of T2 and T2 data, (n=6).

FIG. 8 depicts the effect of stressed NPCs (sNPC) on pain outcomes in healthy IVDs in vivo. The results of the biobehavioral tests (BBTs) show that intradiscal injection of stressed NPCs can result in behavioral signs of pain. 2-way RM-ANOVA #p<0.05, ##p<0.01, ###p<0.001 (compared to BL), *p<0.05, ** p<0.01, tp<0.1 (group comparison, human NPC vs saline).

FIG. 9 depicts LBP phenotype in SPARC null mice, a genetic model of accelerated IVD degeneration and LBP presents: (panel A) increased sensitivity to cooling stimuli that is attenuated by anti-neuropathic but not anti-nociceptive or anti-inflammatory drugs. (panel B) decreased grip force strength, indicative of radiating leg pain and axial discomfort, respectively. (panel C) Top: SPARC-null mice do not differ from WT mice in their average speed during the first 5 minutes of the FlexMaze test but are significantly slower than WT mice during the next 5 minutes (Prior Art, adopted from Millecamps, M., et al., Pain 153, 1167-79 (2012)); Bottom: a schematic illustrating the design of the FlexMaze. Two chambers are connected via a maze with staggered doors.

FIG. 10 depicts the transfection of BM-MSCs using MaxCyte technology. Human BM-MSCs were transfected with GFP reporter using 3 different energy settings of electroporation (EP). GFP expression was analyzed using flow cytometry 24 hours post-transfection.

FIG. 11 depicts neonatal vs adult human intervertebral disc (IVD) cells analysis using scRNAseq. (Panel A) We isolated single cells from neonatal and adult IVD, then run through 10× Genomics' scRNAseq workflow for comparing single cell gene expression. (Panel B) Uniform Manifold Approximation and Projection (UMAP) of neonatal and adult samples identified 14 cell populations categorized into 5 major types as shown in the inlet. Markers were shown for each cell population. (Panel C) Dot plots show the overexpression of classical markers for each cell population. (Panel D) Pseudo-time trajectory shows the developmental directions in neonatal and adult samples.

FIG. 12A-12C depict stepwise differentiation of iPSC to iNC in vitro. (12A) Step 1: Differentiation of (e.g., human fibroblast-derived) iPSCs into primitive streak (PS) cells (or primitive streak mesoderm (PSM) cells). Microscopic images show the morphological changes during GSK3i treatment; and gene expression analysis of PS cells shows a rapid decline in expression of pluripotency marker (Nanog, Oc4 and Sox2) and increase in mesodermal markers (MIXL1, BR, FoxF1) in GSK3i-treated compared to DMSO-treated cells. Results were calibrated relatively to iPSCs (Day 0). (12B) Step 2: Differentiation of PS cells to iNC progenitors by overexpression of Brachyury (Br) transcription factor, wherein the PS cells were transfected with Br-encoding plasmid. On Day 2, 4, and 6 gene expression analysis was performed and showed a rapid reduction in notochordal markers in both groups, but elevation in the transfected genes with time. N=6, bars indicate SEs. (2C) Step 3: Maturation of iNC progenitors into iNCs in NP-like environment and paracrine effect on BM-MSCs. PS-Br Day 2 cells (iNC progenitors) were embedded in TETRONIC1307-Fibrinogen (TF) gel (a 3D culture), cultured in NP-specific media in hypoxic condition (e.g., 2% O₂), i.e., an NP-like or simulated NP environment, for up to 8 weeks. Briefly, the cells could be seeded in 1-kPa hydrogels containing 0.1% w/v Irgacure 2959 initiator (Ciba) at a seeding density of 3×10⁶ cells/ml; and the hydrogels were incubated for about 5 min at 4° C. and then cross-linked under long-wave UV light (e.g., 365 nm, 4e5 mW/cm²) for about 8 min. “NP-specific media” is a culture medium suitable for NP tissue culture, such as an NP differentiation medium, or notochordal cell conditioned medium (NCCM). NP differentiation medium can be: DMEM/F-12 with 15 mM HEPES, L-glutamine, and pyridoxine hydrochloride (1:1, v/v; Life Technologies), with additional L-ascorbic acid-2-phosphate (sterilized using a 0.22 μm filter; Sigma), non-essential amino acids, insulin transferrin-selenium (ITS) and penicillin-streptomycin (all from Life Technologies). NCCM can be media collected from immature NP tissue explant culture: immature porcine NP tissues containing largely notochordal-like cells were incubated in DMEM-based culture media under hypoxic conditions for 4 days, and at the end of the incubation, the conditioned media was collected, concentrated, and stored at −80° C. until used as a supplement to the cell (e.g., iPSC) culture. Additionally, the iNCs were mixed with BM-MSCs and co-cultured in TF gels in order to test the paracrine effect of iNCs on BM-MSCs. As a control, BM-MSCs were cultured alone in the same settings. Every 2 weeks, TF gels were extracted and tested for qRT-PCR. Results show retention to the NC phenotype once cultured in NP environment. Results are presented as mean RQs calibrated to PS cells (Day 0). Immunofluorescence staining was performed for NC markers (e.g., BASP1) and NP markers (e.g., CTGF and CD24). Both NP markers were expressed in BM-MSCs and less in iNCs. iNCs were further injected in saline into a healthy IVDs isolated as organ culture. The cells were found to survive for at least 2 months ex vivo as demonstrated by histology and Live/Dead assay. (Prior Art, modified from Sheyn et al., Theranostics 2019, vol. 9, issue 25.)

FIG. 13 is a schematic depicting some experimental design in Example 5.

FIG. 14 is a schematic depicting in vivo experimental design in Example 5. Post harvest, iNC-GFP injected discs will be used only for GFP+ cell isolation and RNAseq. “Hydrogel only” and iNC-Dil discs will be cut in half, one half will be used for immunoassays (ELISA) for NP matrix component analysis, the other half will be used either for histology and immunofluorescence or for protein extraction and DMMB assay.

FIG. 15A-15N depict characterization and efficacy of iNC-loaded microgels as low back pain therapy. (15A, 15B) microfluidic device. (15C) Phase image of FF cell-loaded microgel in the device. (15D) Fluorescent image of DiD-labeled cells encapsulated in FF microgels at 10 million cells/1 mL microgel suspension, scale bar=300 μm. (15E) The ferret size (diameter) of microgels generated in microfluidic device and after a purification step to remove the oil phase, measured using ImageJ, **** p<0.0001. (15F) Rheological property of FF microgel: storage modulus (G′) and loss modulus (G″) changed with temperature heating ramp. (15G) Upper graph: viability of microgel-encapsulated cells after 1 week of pre-conditioning culturing. Cells in bulk gel served as a control group. Lower graph: iNCs are more viable than bulk gels over 5 weeks of pre-conditioning culturing (in vitro). iNCs were encapsulated in FF microgels at 10 million cells/1 mL gel density. Cell viability assays (Cellglo) were performed on samples at day 0 (DO), week 1, 2, 3, and 5 (w1, w2, w3, w5). Bulk gel was used as a control for comparison. Specifically, 10 million cells/1 mL cell/gel solutions were loaded into syringe, and injected directly into warm media. The amount of injected gels is the same as in the microgel group. * p<0.05 compared to DO. #p<0.05 compared within the same time point. (15H) Immunostaining of DiD labeled iNCs with Col2 antibodies on Day 1 after encapsulation; and (15I) immunostaining of DiD labeled iNCs with Col2 antibodies on day 14, imaged by confocal microscopy. (15J) Posterior view of 21G needle used to induce disc degeneration percutaneously. (15K) Experimental design of the in vivo study. The iNC-microgels were compared to saline control. (15L) Acetone cold sensitivity result. (15M) von Frey biobehavioral tests indicating nociceptive behaviors (n=7; * p<0.05, ** p<0.01 between groups, ##p<0.01 compared to baseline). (15N) Disc height quantification at baseline (W-2), 2 weeks post injury (W0) and 2 weeks post treatment (W2).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Polyoxyalkylene” refers to an oligomer or polymer of an oxyalkylene, or —O(CH₂)n— group, where n is in the range of 1 to 10 and where any H may be substituted for a linear or branched alkyl group. In preferred embodiments, n is 2 or 3, and is either unsubstituted or substituted by methyl group. In various embodiments, the polyoxyalkylene comprises segment of hydrophobic character, e.g., poly(oxypropylene) blocks, and segment of hydrophilic character, e.g., poly(oxyethylene) blocks, in order to facilitate aggregation. In various embodiments, the polyoxyalkylene is a poloxamer (PLURONIC®), or poly(ethylene glycol)-block-poly(propylene glycol)-block-poly(ethylene glycol).

The generic term “poloxamers” are commonly named with the letter “P” (for poloxamer) followed by three digits, the first two digits×100 give the approximate molecular mass of the polyoxypropylene core, and the last digit×10 gives the percentage polyoxyethylene content (for example, P407-Poloxamer with a polyoxypropylene molecular mass of 4,000 g/mol and a 70% polyoxyethylene content). In certain embodiments, the poloxamer may comprise a polyoxypropylene molecular mass in the range of 2,000 to 6,000 g/mol; in further embodiments, the polyoxypropylene molecular mass may be in the range of 2,500 to 5,000 g/mol. Additionally, the poloxoamer may have from 30% to 90% polyoxyethylene content; in further embodiments, the poloxamer may have a polyoxyethylene in the range of 60% to 80%.

For the PLURONIC tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content). For example, poloxamer 181 (P181) is equivalent to Pluronic L61.

“Poloxamines” (TETRONIC®) are X-shaped amphiphilic block copolymers formed by four arms of poly(ethylene oxide)-poly(propylene oxide) (PEO-PPO) blocks bonded to a central ethylenediamine moiety.

A polymer is created via polymerization of monomers, and can also be referred to in some embodiments as a polymer derived from a monomer.

A polymeric segment is part of a larger molecule, and a polymeric segment derived from polyoxyalkylene (or another compound) refers to polyoxyalkylene (or the other compound) with at least a valence electron for bonding with another segment of the larger molecule, thereby forming the larger molecule. In various embodiments of the composition of matter disclosed herein, a polymeric segment derived from polyoxyalkylene is bonded with a polymeric segment derived from a polypeptide or polysaccharide, thereby forming a macromolecule that is a copolymer or hybrid polymer, which in a quantity forms a polymeric network. In various embodiments, a polymeric segment derived from polyoxyalkylene has a valency of at least two, and a polymeric segment derived from a polypeptide or polysaccharide has a valency of at least one; so that bonding of a plurality of the polymeric segment derived from the polyoxyalkylene with a plurality of the polymeric segment derived from the polypeptide or polysaccharide forms a cross-linked polymeric network.

“Gelation” or “viscosification” refers to a drastic increase in the viscosity of the polymer solution. Gelation is dependent on the initial viscosity of the solution, but typically a viscosity increase at about pH 7 and 1 wt % polymer concentration is in the range of preferably 2- to 100-fold, and preferably 5- to 50-fold, and more preferably 10- to 20-fold for a composition which is used in the preparation of the compositions of the invention. Such effects are observed in a simple polymeric solution and the effect may be modified by the presence of other components in the final composition.

A process of reversibly gelling/gelation takes place upon an increase in temperature rather than a decrease in temperature. This is counter-intuitive, since solution viscosity typically decreases with an increase in temperature. A reversible gel refers to gels comprising components that have the capacity to make, break, and modify the bonds responsible for holding the network together. For example, poloxamers forms a thermoreversible gel. Without wishing to be bound by a particular theory, at low temperatures in aqueous solutions, a hydration layer surrounds poloxamer molecules and hydrophobic portions are separated due to hydrogen bonding; and when the temperature is raised, the hydrophilic chains of the copolymer become dehydrated as a result of the breakage of the hydrogen bonds. This results into hydrophobic interactions amongst the polyoxypropylene domains and gel gets formed when concentration is above critical micellar concentration. In contrast, other gels held together by covalent bonds do not have this capability.

“Microgel,” “microgel particle,” “gel microparticle,” “hydrogel microparticle,” “hydrogel microsphere,” and “microsphere” refer to a particle in the micron size range, which comprises a plurality of cross-linked monomers or polymeric polypeptide/polysaccharide segments, which have formed a polymer network throughout each particle as a result of a polymerization reaction. A micron size can be a dimension between 1 μm and 1000 μm, preferably between 30 μm and 500 μm, or more preferably between 50 μm and 300 μm. In some embodiments, a polymerization will have been conducted during the preparation of a particle. The microgel particle is preferably a cross-linked polymer particle that undergoes a conformation change and forms a gel (or microgel) in response to an environmental stimulus, such as an increase in temperature, exposure to irradiation by UV or visible light, and/or change in pH. In other embodiments, a polymerization occurs in situ, e.g., after administration into a subject. In some embodiments, “microgel,” “gel microparticle,” “microgel particle,” and “hydrogel microparticle” are used interchangeably, which is in a spherical or near spherical shape, and hence also referred to as microsphere or hydrogel microsphere. In other embodiments, “microgel,” “gel microparticle,” “microgel particle,” and “hydrogel microparticle” are used interchangeably, which is in any shape having a dimension between 1 μm and 1000 μm, for example a disc shape or a cube shape.

“Polysaccharide” refers to a polymeric carbohydrate having a chemical structure formed of repeating units including mono-saccharides or di-saccharides joined together by glycosidic bonds. The polysaccharide may be linear or branched, homopolysaccharide or heteropolysaccharides. The polysaccharides may be amorphous or crystalline. The term “polysaccharide” includes polysaccharides that have been modified by a reaction of its hydroxyl groups or other group with a compound to a different pendent functional group. Exemplary polysaccharides include but are not limited to hyaluronic acid, chitosan, cellulose, dextran, glucan, and their derivatives, especially derivatives in the form of ester and ether. Bioadhesive polysaccharides include polysaccharides with innate ability for mammalian cells to adhere to and those modified with peptides that facilitate mammalian cell adhesion, such as sequence comprising contiguous amino acids of RGD.

“Statistically significant” generally means that the difference between two values has a p-value of ≤0.05, i.e., has a 95% or higher chance of representing a meaningful difference between the two values. Hence, “not statistically significantly different” means the difference between two values has a p-value of >0.05.

In contrast to analgesic treatments, such as opioids that are frequently prescribed to relief chronic discogenic pain, our treatment approach, using microgel/microtissue embedded stem cells, is potentially disease-modifying and not associated with the risk of developing drug addiction. We previously developed a protocol for stem cell differentiation and demonstrated the cells' efficacy in reduction of IVD degeneration in a large animal model (US20200093961 and Sheyn, D. et al. Theranostics 9, 7506-7524 (2019), which are herein incorporated by reference in their entireties). Herein, we improve the delivery system of the cell candidate by embedding them in microgels/microtissues, evaluate its therapeutic potential to reduce discogenic pain by performing efficacy and reproducibility studies, and prepare it for IND-enabling studies and clinical trials, for treating, mitigating, or providing interventions to one the most common musculoskeletal disorders and taking huge population of patients off opioids. Herein, we microencapsulate iPSC-derived stem cells in microgels/microtissues for improvement in the stem cell preparation and delivery in clinical settings through development of biomanufacturing-ready protocols and preparation for the translational stage of this study. We combine iPSCs-derived NCs with an injectable carrier comprising microgel particles, optionally crosslinked in situ after injection, and the microgel particles will not only support the cell viability and differentiation, but also provide the necessary biomechanical stiffness. Unless otherwise noted, the gelation and/or crosslink is within microparticle gelation/crosslink, so as to form microgel. Furthermore, we use advanced behavioral studies and single cell transcriptomic analysis, to determine cell efficacy and identity to evaluate the cell therapeutic impact and to unravel the mechanism of action of our candidate.

Microgels provide a 3D environment for iNCs, appropriate biomechanical properties, a low cellular density, and protect the cells from the harsh environment of the degenerated IVD. We conceive that iNCs embedded into microgels/microtissues can be injected to fill the degenerated IVD, and attenuate disc degeneration, reduce discogenic LBP, and eventually facilitate disc rejuvenation, and that preconditioning iNC-loaded microgels (resulting in extracellular matrix protein deposition, hence iNC-laden microtissues) will enhance the cell activity and viability and therefore will enhance the host integration of iNCs and their therapeutic potential for both attenuation of disc degeneration and rejuvenation of IVD, compared to bulk hydrogel injections. This treatment is a minimally invasive approach while allowing for optimized cell differentiation and mechanical strength.

Various embodiments provide injectable compositions, wherein a microgel and an iPSC-derived notochordal cell (iNC) are included, and the iNC is encapsulated in the microgel. In various aspects, an injectable composition includes a quantity of the microgel and over 50%, 60%, 70%, 80%, or 90% of the quantity of the microgel contain at least one iNC (or more preferably two or more iNCs, such as more than five, ten, 20, 30 or 50) in each. In various aspects, an injectable composition includes a dispersion comprising microgel particles and human iPSC-derived notochordal cells (iNCs), wherein the iNCs are encapsulated in the microgel particles, and the size of the microgel particles is between 30 μm and 1000 μm. In various aspects, an injectable composition including a dispersion of microgel particles encapsulating human iNCs is featured with one or more of: (1) one or more extracellular matrix proteins, e.g., collagen or collagen type 2, are expressed by the iNCs and present in the microgel particles, (2) a storage modulus (G′) of at least 100 Pa at a temperature of about 25° C. or higher, and (3) a viability of the encapsulated iNCs at 1 week (or 7 days after encapsulation) being statistically similar to baseline (at time of or right before encapsulation). For example, at least 100%, 90%, 80%, 70%, 60%, or 50% of viability (or cell number) of the encapsulated iNCs in the microgel particles at 1 week, 2 weeks, 3 weeks, 4 weeks, or 5 weeks compared to baseline, or the viability of the encapsulated iNCs at 1 week, 2 weeks, 3 weeks, and/or 4 weeks are not statistically significantly different compared to baseline.

In various embodiments, the microgel particles each comprises a cross-linked polymeric network comprising: a plurality of first polymeric segments derived from a polyoxyalkylene, and a plurality of second polymeric segments derived from a bioadhesive polypeptide or polysaccharide, wherein the first polymeric segments and the second polymeric segments are bonded together to form a polymeric network.

In various embodiments, the first polymeric segments are reversible gelling materials, preferably thermoreversible gelling materials, and as a result, a hybrid copolymer including the first polymeric segment and the second polymeric segment is a reversible gelling copolymer.

In some embodiments, the hybrid copolymer comprises at least a first block/segment comprising a polyoxyalkylene, which preferably has a hydrophobic region and a hydrophilic region, and a second block/segment comprising a protein/polypeptide or polymer (such as polysaccharide), wherein the first block/segment and the second block/segment are bonded together. Alternatively, the polyoxyalkylene, preferably a thermally gelling polymer, and the polypeptide or polysaccharide are combined in a blend (e.g., a mixture). In further aspects, the first polymeric segments, the second polymeric segments, or the molecules of them when not bonded, are independently functionalized with a photo-reactive chemical group. When functionalized with a photo-reactive chemical group, the first polymeric segments, the second polymeric segments, or the molecules of them may further be photo-cured or crosslinked.

In some embodiments, the first polymeric segments of the polymeric network comprise or are derived from a polyoxyalkylene which is a poloxamer, and the poloxamer consists of or includes a central hydrophobic block of polyoxypropylene flanked by two hydrophilic blocks of polyoxyethylene. In one embodiment, the approximate length of the propylene glycol block is between about 35-65 repeat units and the approximate length of the PEG blocks is between about 75-125 repeat units. In one embodiment, the approximate weight of the propylene glycol block is between about 3,000 and 5,000 g/mol and the approximate percentage of polyoxyethylene content is between about 50% and 90%. In one embodiment, the poloxamer is PLURONIC F127 or poloxamer 407.

In other embodiments, the first polymeric segments of the polymeric network comprise or are derived from a polyoxyalkylene which is polyethylene glycol (PEG).

In some embodiments, the second polymeric segments of the polymeric network comprise or are derived from polypeptides or polysaccharides, preferably bioadhesive ones either as the polypeptides' or polysaccharides' innate property or with modification of an adhesion peptide. Exemplary polypeptides or polysaccharides for forming a polymeric network of the microgel particles include but are not limited to fibrinogen, fibrin, laminin, hyaluronic acid, cellulose, chitosan, dextran, glucan, or derivatives thereof. In some embodiment, the polypeptide is or comprises fibrinogen. In some embodiment, the polypeptide is or comprises laminin. In some embodiment, the polypeptide is or comprises hyaluronic acid.

The hybrid copolymer or the resultant crosslinked polymeric network may be produced from any desired ratio of the first polymeric segment (e.g., polyoxyalkylene, preferably poloxamer or poloxamine) to the polypeptide or polysaccharide. The weight ratio of the polyoxyalkylene to the polypeptide or polysaccharide may be from 1:99 to 99:1. In some embodiments, the ratio of poloxamer or polyoxyalkylene to bioadhesive polypeptide or polysaccharide in forming the microgel may be from 30:70 to 70:30. In some embodiments, the weight ratio of the polyoxyalkylene to the polypeptide or polysaccharide is between 1:99 and 10:90. In some embodiments, the weight ratio of the polyoxyalkylene to the polypeptide or polysaccharide is between 10:90 and 20:80. In some embodiments, the weight ratio of the polyoxyalkylene to the polypeptide or polysaccharide is between 20:80 and 30:70. In some embodiments, the weight ratio of the polyoxyalkylene to the polypeptide or polysaccharide is between 30:70 and 40:60. In some embodiments, the weight ratio of the polyoxyalkylene to the polypeptide or polysaccharide is between 40:60 and 50:50. In some embodiments, the weight ratio of the polyoxyalkylene to the polypeptide or polysaccharide is between 50:50 and 60:40. In some embodiments, the weight ratio of the polyoxyalkylene to the polypeptide or polysaccharide is between 60:40 and 70:30. In some embodiments, the weight ratio of the polyoxyalkylene to the polypeptide or polysaccharide is between 70:30 and 80:20. In some embodiments, the weight ratio of the polyoxyalkylene to the polypeptide or polysaccharide is between 80:20 and 90:10. In some embodiments, the weight ratio of the polyoxyalkylene to the polypeptide or polysaccharide is between 90:10 and 99:1.

In some embodiments, the polymeric network includes linking groups connecting the first polymeric segments to the second polymeric segments. Hence, in some embodiments, the polymeric network is chemically cross-linked. For example, the polyoxyalkylene or poloxamer or poloxamine can be functionalized with a first cross-linkable functional group (e.g., in a quantity of two or more per polyoxyalkylene) and the bioadhesive polypeptide or polysaccharide can be functionalized with a complementary cross-linkable functional group (e.g., in a quantity of one or two or more per polypeptide or polysaccharide). A complementary cross-linkable functional group may be any group that can react or otherwise form a bond or linking group between the polymeric segments. Preferably when included, the linking groups are biocompatible linking groups in the polymeric network, which would not include functional groups that show significant toxicity to the patient either in the polymeric form or the residues of biodegradation.

In some embodiments, the linking groups may comprise ester groups. In some embodiments, the polyoxyalkylene (e.g., poloxamer or poloxamine or PEG) is functionalized with two or more acrylate groups such as methacrylate, and the polypeptide or polysaccharide is treated with a reducing agent to present a thiol group or modified with a thiol group. In some embodiments, both the polyoxyalkylene (e.g., poloxamer or poloxamine or PEG) and the polypeptide or polysaccharide are functionalized with cross-linkable double bonds or photo-reactive chemical groups. Crosslinking may then be conducted via radical polymerizations, UV initiated cross-linking, e-beam curing, or other polymerization process.

Exemplary photo-reactive chemical groups include but are not limited to an acryloyl group, an acrylate, an aryl azide, an azido-methyl-coumarin, a benzophenone, an anthraquinone, a diazo, a diazirine, or a psoralen. When exposed to ultraviolet, visible light or another irradiation, the chemical groups become reactive and the block/hybrid copolymer is bonded or crosslinked to form hydrogel or a microgel if in the shape of a micron-sized particle. In some embodiments, the polymer or a segment thereof was modified with an acrylate group, which was photo-reactive and also suitable for the thiol-acrylate Michael addition, so that the polymer is formed (e.g., with a linking group such as an ester) resulting from the acrylate-modified polymeric segment (e.g., via the thiol-acrylate Michael addition).

Preferably, the first polymeric segments are based on reversibly gelling compositions (e.g., thermally gelling polymer). A material with this property is poloxamers. See. U.S. Pat. Nos. 4,188,373, 4,478,822 and 4,474,751, where are incorporated by reference. Adjusting the temperature of the polymer gives the desired liquid-gel transition. Another material which is liquid at room temperature but forms a semi-solid when warmed to about body temperature is poloxamines, which are formed from tetrafunctional block polymers of polyoxyethylene and polyoxypropylene, condensed with ethylenediamine. See, U.S. Pat. No. 5,252,318, which is incorporated by reference herein.

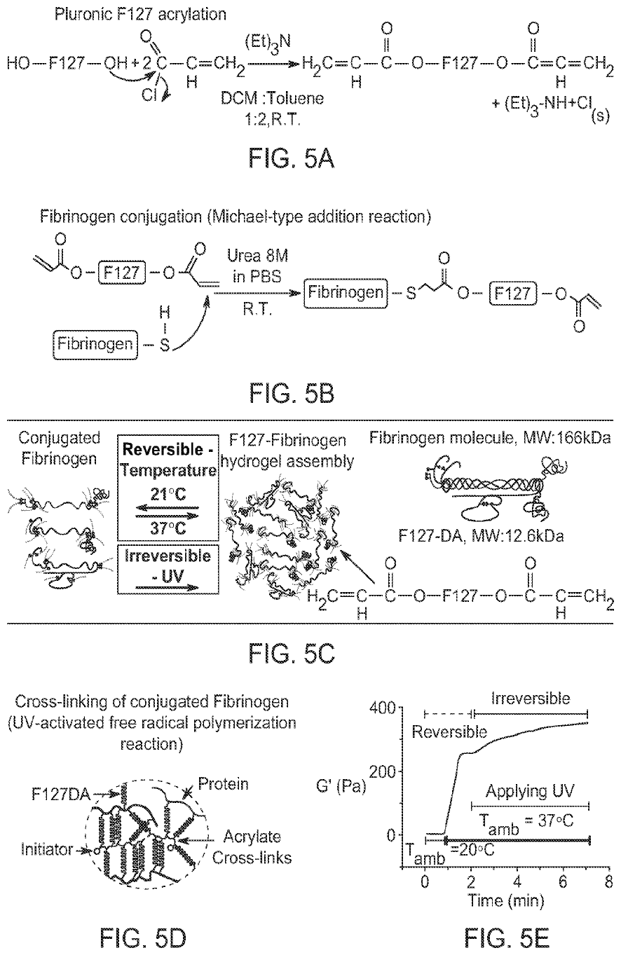

In some embodiments, the polymeric network of the microgel particles comprises a hybrid copolymer comprising or consisting essentially of a first block comprising poly(ethylene oxide)-b-poly(propylene oxide)-b-poly(ethylene oxide) and a second block comprising fibrinogen. In some embodiment, this hybrid copolymer is an adduct formed between an acrylate group-modified poly(ethylene oxide)-b-poly(propylene oxide)-b-poly(ethylene oxide) and a disulfide-reduced/thiol group-containing fibrinogen. The adduct is formed via a ‘click’ Michael-type addition chemistry between acrylate groups and thiol groups. For example, a PLURONIC or TETRONIC poloxamer is modified with bi- or multiple acrylate groups, and fibrinogen is reduced to present a thiol group, such that the modified poloxamer or poloxamine and the reduced fibrinogen form an adduct, such as a crosslinked adduct, see FIG. 5.

In some embodiments, freshly differentiated iNCs are microencapsulated in thermal responsive (e.g., Fibrinogen-F127 (FF)) microgel particles, for example, using a microfluidic system. For example, iNCs are encapsulated in microgels formed from fibrinogen-poloxamer adduct (e.g., poloxamer being FF127) having an FF concentration between 8 and 15 mg/mL. In some embodiments, iNCs are encapsulated in microgel formed from fibrinogen-poloxamer adduct having a poloxamer concentration of 10-13 mg/mL. In some embodiments, iNCs are encapsulated in microgel formed from fibrinogen-poloxamer adduct having a poloxamer concentration of 11-12 mg/Ml. In some embodiments, iNCs are encapsulated in microgel formed from fibrinogen-poloxamer adduct having a poloxamer concentration of about 11.7 mg/mL. Preferably, fibrinogen-F127 microgels encapsulate the iNCs at a cell density of about 1×10⁶/mL of microgel dispersion. Preferably, the iNCs are encapsulated in microgel particles in a number ratio between 1:1 and 80:1. In some embodiments, microtissue is provided based on iNC-loaded microgels cultured for 3-21 days or longer for matrix deposition. In some embodiments, iNC-loaded microgels are cultured in hypoxic culture condition (i.e., a low oxygen environment that is under normoxic, 21% oxygen tension condition, for example a low oxygen environment of 10% or less, between 1% and 5% oxygen) for 5-20 days, so that extracellular matrix protein(s) are expressed and deposited in the microgels. In some embodiments, iNC-loaded microgels are cultured in hypoxic culture condition (i.e., a low oxygen environment that is under normoxic, normoxic being about 21% or 20.9% oxygen tension condition, for example a low oxygen environment of 10% or less, between 1% and 5% oxygen) for 6-18 days. In some embodiments, iNC-loaded microgels are cultured in hypoxic culture condition (i.e., a low oxygen environment that is under normoxic, 21% oxygen tension condition, for example a low oxygen environment of 10% or less, between 1% and 5% oxygen) for 7-14 days. In some embodiments, a hypoxic culture condition is one with oxygen content between 1% and 5% in total gas mixture. In some embodiments, a hypoxic culture condition is one with oxygen content between 2% and 4% in total gas mixture. In some embodiments, a hypoxic culture condition is one with oxygen content between 5% and 10% in total gas mixture. In some embodiments, a hypoxic culture condition is one with oxygen content between 10% and 15% in total gas mixture. In further embodiments, the microtissue formed from iNC-loaded microgels is in a disc shape, e.g., the iNC-loaded microgels are molded in a disc shape. Alternatively, the microtissue formed from iNC-loaded microgels is injected into degenerative intervertebral discs (or the nucleus pulposus area of intervertebral discs). In other embodiments, iNCs mixed with bulk hydrogel prepared from a fibrinogen-PLURONIC®F127 adduct are provided, and the bulk hydrogel can be in a disc shape. Thermoresponsive hydrogels have the advantage that they do not require chemical or UV-activated crosslinkers and are relatively easy to scale for biofabrication. The pre-conditioning culture of microgel-embedded cells in vitro allows for extracellular matrix secretion and formation of microtissues.

In some embodiments, iNCs are encapsulated in PEG-fibrinogen microgel particles, and the microparticles may further be crosslinked (intraparticle crosslinked) in situ after injection to intervertebral disc. In some embodiments, PEG-fibrinogen hydrogel is prepared by a process where fibrinogen fragments are PEGylated with PEG-diacrylates, mixed with photoinitiator and exposed to UV light to form a hydrogel material in the presence of a cell suspension, for encapsulation of iNCs.

In some embodiments, iNCs are encapsulated in microspheres prepared from hydrogel that is functionalized with laminin, such as laminin functionalized poly(ethylene glycol) (PEG-LM111) hydrogel. In some embodiments, laminin-111 is PEGylated with acrylate-PEGN-hydroxysuccinimide to introduce functional acrylate groups for crosslinking. Precursor PEG-LM111 conjugate solutions can be purified by dialysis to remove any unreacted Ac-PEG-NHS. And PEG-LM111 conjugate solutions can further dissolve PEG-octoacrylate and PEG-dithiol, and hydrogel forms upon thiol-acrylate Michael addition reaction.

In some embodiments, iNCs are encapsulated in microspheres prepared from hyaluronic acid (HA)-based hydrogel, or high molecular weight HA-based hydrogel. In some embodiments, HA cross-linking is realized using a multi-arm (e.g., 4-arm) PEG-amine, in which free carboxyl groups of HA and free amine groups of PEG-amine are reacted.

Some embodiments provide that iNC is prepared by a process including:
culturing iPSCs (e.g., human iPSCs) in the presence of a glycogen synthase kinase 3 (GSK3) inhibitor (GSK3i) to form primitive streak (PS) cells;
transfecting the PS cells with a vector encoding Brachyury to overexpress Brachyury;
expressing Brachyury in the PS cells, wherein expression of Brachyury by the vector encoding Brachyury in the PS cells induces formation of iNCs (e.g., human iNCs), and the iNCs express Brachyury, Keratin 18, and Keratin 19.

In some embodiments, the human iPSCs are cultured in the presence of at least 2 μM, or 3-8 μM, or 4-6 μM GSK3 inhibitor for at least 1 day. In some embodiments, the human iPSCs are cultured in the presence of 4-6 μM GSK3 inhibitor for at least 1 day and up to 6 days. Exemplary GSK3 inhibitors include but are not limited to CHIR-99021 (laduviglusib), SB216763, AT7519, CHIR-98014, TWS119, tideglusib, SB415286, AZD2858, AZD1080, AR-A014418, TDZD-8, LY2090314, WAY-119064, PF-04802367, (E/Z)-GSK-3β inhibitor 1, KY19382, BRD0705, alsterpaullone, BIO-acetoxime, IM-12, 1-azakenpaullone, indirubin, indirubin-3′-oxime, resibufogenin, elraglusib, 5-bromoindole, CP21R7, or bikinin. Alternative, optional, and/or complementary steps of preparing iNCs are described in US20200093961 or U.S. Pat. No. 11,554,195, which are incorporated by reference herein in its entirety.

In some embodiments, iPSCs are obtained as autologous stem cells reprogrammed from the somatic cells of the patient. In other embodiments, iPSCs are obtained as allogeneic cells. In some embodiments, human iPSCs are derived from human fibroblasts. In some aspects, an allogeneic cell source is attractive since the IVD is considered immunoprivileged and HLA matching repositories are being established all over the world. For example, iNCs are generated from human induced pluripotent stem cells obtained from blood samples from donors. In some embodiments, iNCs are derived from iPSC lines of different HLA types and diverse genetic backgrounds using e.g. single-cell RNA sequencing to improve compatibility with the patient's immune system. HLA (human leukocyte antigen) are proteins expressed on the cell surface that allow the immune system to distinguish self and foreign cells; in which class I (HLA-A, -B, and -C) out of the three classes is considered most important when donor-patient matching.

Further embodiments provide that the injectable compositions have been cultured in a medium for a sufficient amount of time for the microencapsulated iNC to (1) exhibit at least about 50%, 60%, 70%, 80%, or 90% activity compared to that when iNC is cultured in the medium without microencapsulation, and/or to (2) secrete extracellular matrix protein(s) in the microgel, preferably collagen or type II collagen, also known as forming a microtissue. In some aspects, an injectable composition has been cultured with the medium (including replenishing volumes of the medium) for at least a week, 2 weeks, 3 weeks, or 4 weeks; or about 5, 6, 7, 8, 9, 10, 12, 14, 16, 18, 20, 22, 24, 26, 28, or 30 days. In some aspects, an injectable composition will be cultured in a medium (including replenishing volumes of the medium) for at least a week, 2 weeks, 3 weeks, or 4 weeks, or about 5, 6, 7, 8, 9, 10, 12, 14, 16, 18, 20, 22, 24, 26, 28, or 30 days, before administration to a subject in need thereof.

In some implementations, human bone marrow-derived mesenchymal stem cells (MSCs) are co-injected with iNCs-loaded microgel particles, or MSCs are co-encapsulated with iNCs in the microgel particles.

In some embodiments, the injectable composition is a pharmaceutical composition, which includes a therapeutically effective amount of microgel particles which encapsulate iNCs, and a pharmaceutically acceptable vehicle. A “therapeutically effective amount” is an amount of iNCs and microgel particles which, when administered to a subject provides prevention and/or treatment of a disease characterized by damaged or degenerated soft tissue (e.g., intervertebral disc). A “subject” may be any vertebrate, mammal, domestic animal or human being.

A “pharmaceutically acceptable vehicle” is any physiological vehicle known to those of ordinary skill in the art useful in formulating pharmaceutical compositions. In a preferred embodiment, the pharmaceutical vehicle is a liquid, and the pharmaceutical composition is in the form of a dispersion.

Various embodiments provide methods for preparing an injectable composition disclosed herein, which include: mixing an aqueous precursor solution for the microgel with the iNC to form a precursor-cell mixture; subjecting the precursor-cell mixture to microinjection/micronization into an oil phase or suspension polymerization, and curing the same by inducing a stimulus effective for inducing gelation of the microsphere, thereby forming a plurality of microgels which encapsulate the iNCs.

In some embodiments, methods for preparing an injectable composition further includes purifying the microgel particles to remove residue from the oil phase.

In some embodiments, the aqueous precursor solution comprises water, a polymer comprising or derived from a polyoxyalkylene and a bioadhesive polypeptide or polysaccharide, wherein the polyoxyalkylene and the polypeptide or polysaccharideare conjugated.

In some embodiments, the environmental stimulus comprises an increase in temperature. In some embodiments, the environmental stimulus comprises an exposure to ultraviolet or visible light, e.g., a beam or a laser beam. In some embodiments, the environmental stimulus comprises an increase in temperature and irradiation/exposure to ultraviolet or visible light.

In some embodiments, the methods for preparing the injectable composition further include culturing the microgel particles that encapsulates the iNCs in cell culture media for a period of time. In some aspects, the period of time, called “preconditioning” period, is at least sufficient for inducing secretion of extracellular matrix by the iNC in the microgel and/or for maintaining of at least 50% activity of the iNC in the microgel compared to before encapsulation. In some embodiments, the pre-conditioning period of culturing is conducted in a hypoxic condition.

In some embodiments of the methods for preparing the injectable composition, the aqueous precursor solution comprises the water and a block copolymer having at least a first block comprising the polyoxyalkylene and a second block comprising the bioadhesive protein or polysaccharide. Optionally, the first block of the block copolymer comprises the polyoxyalkylene having a hydrophobic region and a hydrophilic region, such that the aqueous precursor solution viscosifies in response to the environmental stimulus, said environmental stimulus comprising the increase in temperature, and the microsphere formed from the precursor-cell mixture is thermal-cured to form the microgel. In further implementations, the block copolymer is functionalized with a photo-reactive chemical group, and wherein the curing step comprises subjecting the microsphere to environmental stimuli (sequentially or concurrently) comprising the increase in temperature and the exposure to ultraviolet or visible light.

In some embodiments of the methods for preparing the injectable composition, the polyoxyalkylene and/or the bioadhesive protein or polysaccharide is functionalized with a photo-reactive chemical group, such that the aqueous precursor solution becomes reactive in response to the environmental stimulus, said environmental stimulus comprising the exposure to ultraviolet or visible light, and the microsphere formed from the precursor-cell mixture is photo-cured to form the microgel. In some aspects, the polyoxyalkylene and/or the bioadhesive protein or polysaccharide, functionalized with the photo-reactive chemical group, is not a thermally gelling polymer. In some aspects, the polymer and/or the bioadhesive protein or polysaccharide, functionalized with the photo-reactive chemical group, is a thermally gelling polymer. Hence in some embodiments, the curing step comprises subjecting the microsphere to sequential environmental stimuli comprising the increase in temperature, followed by the exposure to ultraviolet or visible light.

In some embodiments, microgel particles are formed via a dual-phase (or at least two phases) and/or emulsion-based technique. In some embodiments, hydrogel microspheres, or microhydrogel, are formed via a dual-phase, microfluidics technique, i.e., via microinjection. For example, iNCs are mixed with pre-polymer solution (or precursor solution) in an aqueous phase, and the aqueous phase is added, or injected via a small-sized nozzle, to an oil phase (i.e., partition in immiscible phase). For example, the injection via the small-sized nozzle can be referred to as microinjection when the small-sized nozzle is a micron-sized one, such as having an orifice diameter or resulting in a droplet whose cross-section has a diameter of 1-10 μm. In another example, the nozzle has an orifice diameter or produces a droplet whose cross-section size is 10-30 μm. In another example, the nozzle has an orifice diameter or produces a droplet whose cross-section size is 30-50 μm. In another example, the nozzle has an orifice diameter or produces a droplet whose cross-section size is 50-70 μm. In another example, the nozzle has an orifice diameter or produces a droplet whose cross-section size is 70-100 μm. In another example, the nozzle has an orifice diameter or produces a droplet whose cross-section size is 100-200 μm. In another example, the nozzle has an orifice diameter or produces a droplet whose cross-section size is 200-300 μm. In another example, the nozzle has an orifice diameter or produces a droplet whose cross-section size is 300-400 μm. In another example, the nozzle has an orifice diameter or produces a droplet whose cross-section size is 400-500 μm. In another example, the nozzle has an orifice diameter or produces a droplet whose cross-section size is 500-600 μm. In another example, the nozzle has an orifice diameter or produces a droplet whose cross-section size is 600-700 μm. In another example, the nozzle has an orifice diameter or produces a droplet whose cross-section size is 700-800 μm. In another example, the nozzle has an orifice diameter or produces a droplet whose cross-section size is 800-900 μm. Upon vortexing or through the microinjection, micelles or microspheres are generated, respectively, which can be further induced for crosslinking via a stimulus such as UV light. Accordingly, a microgel as described herein may have a spherical or near spherical shape, having a diameter of about 1-10 μm. In some embodiments, a microgel has a diameter or size of 10-30 μm. In some embodiments, a microgel has a diameter or size of 30-50 μm. In some embodiments, a microgel has a diameter or size of 50-70 μm. In some embodiments, a microgel has a diameter or size of 70-100 μm. In some embodiments, a microgel has a diameter or size of 100-200 μm. In some embodiments, a microgel has a diameter or size of 200-300 μm. In some embodiments, a microgel has a diameter or size of 300-400 μm. In some embodiments, a microgel has a diameter or size of 400-500 μm. In some embodiments, a microgel has a diameter or size of 500-600 μm. In some embodiments, a microgel has a diameter or size of 600-700 μm. In some embodiments, a microgel has a diameter or size of 700-800 μm. In some embodiments, a microgel has a diameter or size of 800-900 μm. In various embodiments, a microgel particle is between 30 and 500 μm. In various embodiments, a microgel particle is between 30 and 500 μm in the ‘oil’ phase or before subsequent exposure to an aqueous solution. In various embodiments, a microgel particle is between about 80 and 300 μm in size. In various embodiments, a microgel particle is between about 80 and 300 μm in size in the ‘oil’ phase or before subsequent exposure to an aqueous solution. An injectable composition including the microgel is one that can go through an injection needle between gauge 14 and gauge 32, without being torn, and more preferably no larger than gauge 16. In some embodiments, the injectable composition is for use with a needle of gauge 16. In some embodiments, the injectable composition is for use with a needle of gauge 18. In some embodiments, the injectable composition is for use with a needle of gauge 20. In some embodiments, the injectable composition is for use with a needle of gauge 22. In some embodiments, the injectable composition is for use with a needle of gauge 23. In some embodiments, the injectable composition is for use with a needle of gauge 25. In some embodiments, the injectable composition is for use with a needle of gauge 27.

The partitions can be flowable within fluid streams. In some cases, the partitions may comprise a porous matrix that is capable of entraining and/or retaining materials within its matrix. The partitions can comprise droplets of aqueous fluid within a non-aqueous continuous phase (e.g., oil phase). The partitions can comprise droplets of a first phase within a second phase, wherein the first and second phases are immiscible.

Various embodiments also provide methods for treating a subject with intervertebral disc degeneration and/or discogenic low back pain, which include administering an effective amount of an injectable composition disclosed herein into a nucleus pulposus tissue of the subject. In various implementations, the injectable composition is administered via intradiscal injection to the nucleus pulposus tissue of the subject. In various embodiments, the injectable compositions disclosed herein are used to treat damaged or degenerated vertebral. In various embodiments, the injectable compositions disclosed herein are used to treat deformity or degenerated intervertebral discs (IVDs). In some embodiments, the method comprises administering the injectable composition directly into the IVD. In some embodiments, the method comprises administering the injectable composition into the nucleus pulposus (NP). Hence, advantageously, no surgery is required using the method. In some embodiments, the composition may be administered directly into clefts, which form when the proteoglycan content in the IVD decreases with age. In some embodiments, the injectable composition is for use in treating intervertebral disc degeneration. In some embodiments, the injectable composition is for use in treating back pain. In some embodiments, the injectable composition is for use in treating low back pain. In some embodiments, the injectable composition is for use in treating sciatica. In some embodiments, the injectable composition is for use in treating cervical spondylosis. In some embodiments, the injectable composition is for use in treating neck pain. In some embodiments, the injectable composition is for use in treating kyphosis. In some embodiments, the injectable composition is for use in treating scoliosis. In some embodiments, the injectable composition is for use in treating spondylolysis. In some embodiments, the injectable composition is for use in treating spondylolisthesis. In some embodiments, the injectable composition is for use in treating prolapsed intervertebral disc. In some embodiments, the injectable composition is for use in follow-up treatment after a failed spine surgery. In some embodiments, the injectable composition is for use in treating spinal instability. The disease condition may be chronic or acute. For example, in some embodiments the injectable composition is for treating chronic back pain. In other embodiments the injectable composition is for treating acute back pain.

In some implementations, a method of treating a subject with IVD degeneration or discogenic lower back pain includes administering more than once the iNC-loaded microgels (or microspheres), for example, two or more injection regimens, spaced out by weeks or months. The final dose and volume will be extrapolated based on anatomical size (rat, pig, human) and data collected from the pre-clinical dose-ranging and efficacy studies or in accordance to other clinical trials involving intradiscal injection. In some implementations, 8-10 μl of injection is injected in rats, 100-150 μl in pigs, and/or approximately 500 μl to 1 ml of iNC-loaded microgels to be injected into human degenerated IVD. In some implementations, 100-200, 200-300, 300-400, 400-500, 500-600, 600-700, 700-800, 800-900, 900-1,000, 1,100-1,200, 1,200-1,300, 1,300-1,400, 1,400-1,500, 1,500-1,600, 1,600-1,700, 1,700-1,800, 1,800-1,900, 1,900-2,000 μL of iNC-loaded microgels (optionally further containing mesenchymal stem cells) are injected into human degenerated IVD. In some embodiments, the iNC-loaded microspheres may have a cell density between 0.1×10⁶ and 5×10⁶/ml, or even between 0.7×10⁷ and 5×10⁷/ml. Preferably, fibrinogen-F127 microgels encapsulate the iNCs at a cell density of about 1×10⁶/mL.

In some embodiments, a method of administering the iNC-encapsulated microgel particles further includes irradiating the nucleus pulposus tissue of the subject and/or the injection site of the subject (e.g., with ultraviolet or visible light) to induce photo-crosslinking within the microgel particles.

In some embodiments, the treatment methods disclosed herein are used in patients in patients with one or more of the following characteristics: (1) adult patients with chronic back pain for at least 3 months, (2) failed conservative management (e.g. physical therapy, steroid injections and/or nerve blocks), (3) predominantly back pain (>50%) over leg pain (3) visual analog scale (VAS) of >3 (at least moderate) for back pain, and (4) evidence of IVD degeneration on Magnetic Resonance Imaging (MRI). These characteristics focus on low back pain patients with discogenic pain. IVD degeneration induced pain markers include COMT, IL-6, CGRP, and BDKRB1, and BDNF, whose protein expression or gene expression can be measured. In some embodiments, the treatment methods are used in patients without additional comorbidities like (1) spondylolisthesis (a condition involving spine instability), (2) scoliosis (curved or twisted spine), (3) gravid status, (4) currently undergoing anti-inflammatory therapy, and (5) comorbidities including active infection, cardiac disease, pulmonary disease, malignant disease, and diabetes. In other embodiments, the treatment methods are used in patients without at least one of these additional comorbidities.

Further embodiments provide that the treatment methods disclosed herein result in a significant reduction of the visual analog scale (VAS) for back pain to 0-3 (mild pain or no pain) or reduction in 5 points from the initial VAS score and the reduction of IVD degeneration, optionally demonstrated by MRI as secondary outcome.

In some embodiments, as a result of administration of the injectable composition to the subject, there is preferably, an increase in disc height and/or an increase in the Young's Modulus of the IVD, and the mechanical strength is effectively restored. Advantageously, this is a minimally invasive method that can fill the interior of irregularly shaped clefts in the IVD.

Additional embodiments provide that the treatment methods further include a step of evaluation, such as subjecting the subject to one or more behavioral tests: mechanical and cold sensitivity tests, grip force assay, the open field and rotarod assays for motor ability.

In various embodiments, the iNCs do not exhibit a tumorigenic potential. For example, iNCs do not show a tumorigenic potential in a teratoma formation assay. Lack of blood vessels in the IVD will also ensure that the injected composition will stay local which will decrease the potential off-target effects.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Studies to Improve and Characterize Deliverability of iNCs Encapsulated in Microgels, Microtissues or Delivered in Bulk Hydrogel as Therapeutic Candidates for Injectable Discogenic LBP Treatment In Vitro

iPSCs will be differentiated into iNCs and different cell delivery techniques will be explored: (1) freshly differentiated cells microencapsulated in synthetic Fibrinogen-F127 (FF) microgel spheres using a microfluidic system (2) iNC-loaded microgels cultured for 21 days to allow the formation of microtissue through matrix deposition, (3) iNC mixed with FF bulk hydrogel in a disc-shaped mold.7 Outcome measures will include cell purity and identity by iNC marker expression analysis, cell viability assays, sterility tests, and evaluation of the material composition, consistency and stability.

iPSCs were differentiated into iNCs using our established protocol. Briefly, iPSC was first generated from fibroblast by plasmid nucleofection of fibroblasts with the episomal plasmid expression of six factors—OCT4, SOX2, KLF4, L-MYC, LIN28, and p53 shRNA in Nucleofector Solution (VPD-1001, Lonza), and cultured the cell/DNA suspension under normal oxygen conditions (5% O₂) during reprogramming for 48 hrs, followed by human iPSC medium containing (i) sodium butyrate; (ii) a glycogen synthase kinase 3β inhibitor of the Wnt/β-catenin signaling pathway (CHIR99021, Millipore, Temecula, CA); (iii) a mitogen-activated protein kinase pathway inhibitor; and (iv) a selective inhibitor of transforming growth factor-β type I receptor ALK5 kinase, type I activin/nodal receptor ALK4, and type I nodal receptor ALK7. Colonies with an embryonic stem/iPSC-like morphology will appear 25 to 31 days later, which can be picked and transferred to layers of a standard hiPSC medium-and-Matrigel™ matrix (BD Biosciences, Pharmingen, CA) for feeder-independent maintenance of hiPSCs in chemically defined mTeSR1 medium (Stem Cell Technologies, Vancouver, British Columbia, Canada) and subsequent expansion and cryopreservation if needed.

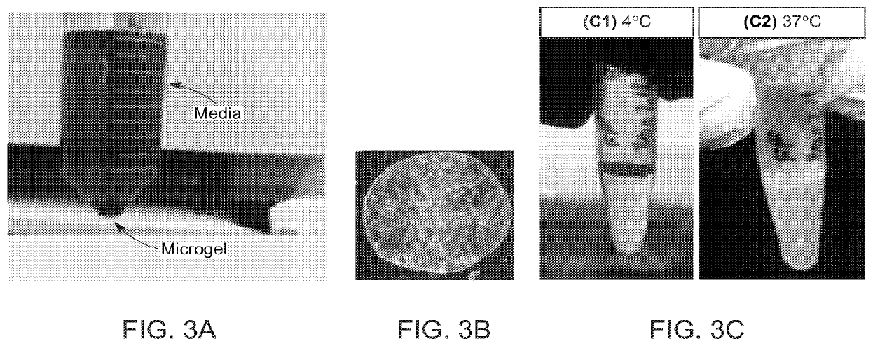

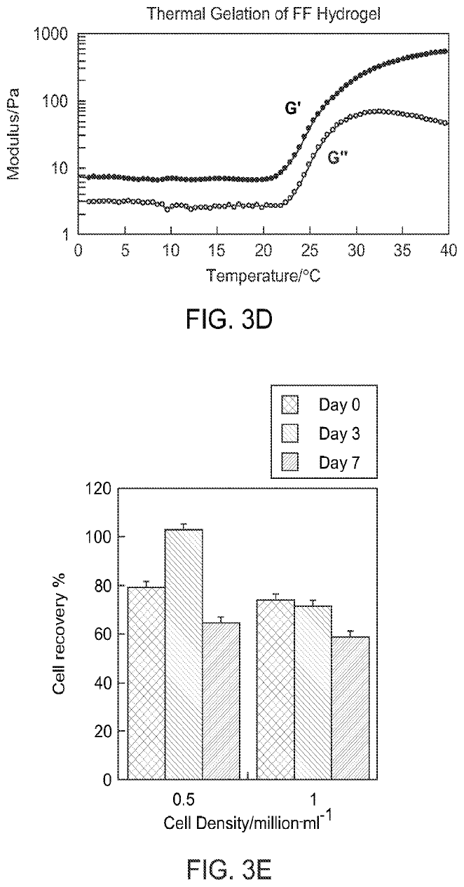

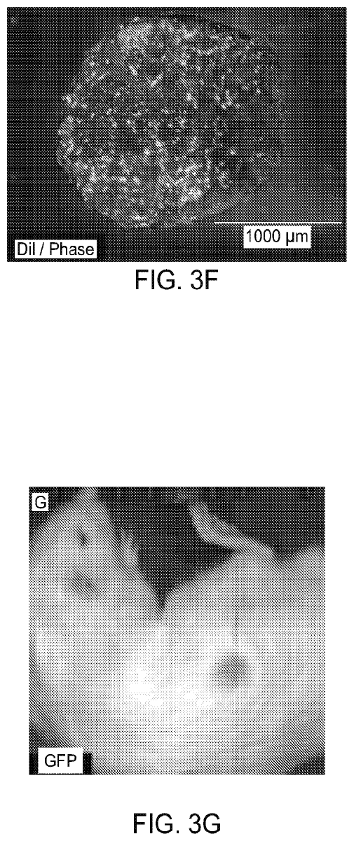

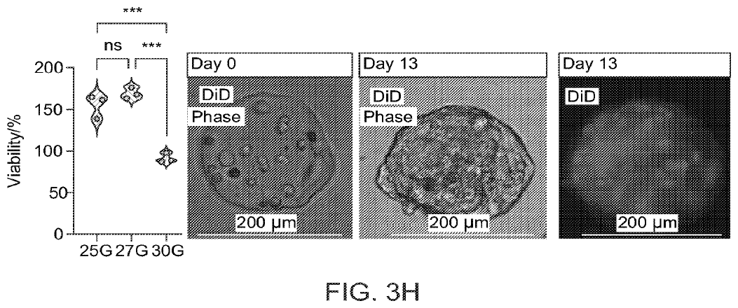

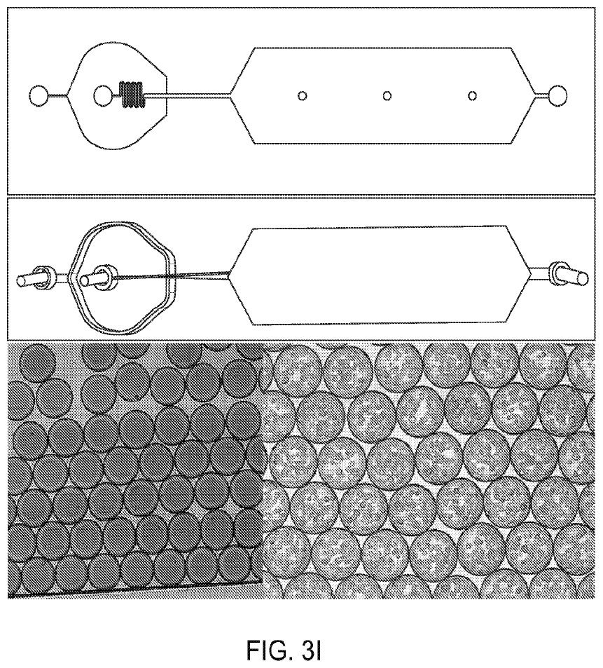

The derivation of iPSC-derived notochordal cells (iNCs) from iPSCs was performed using a 3-step protocol. During Step 1, the iPSCs were differentiated into Primitive Streak Mesoderm (PSM) cells via a 3-day exposure to 5 μM GSK3 inhibitor (e.g., CHIR99021). The media was replaced every 24 hours supplemented with fresh 5 μM GSK3 reconstituted in Dimethyl sulfoxide (DMSO). During Step 2, the GSK3i-treated cells were transfected using Nucleofection technology (Lonza, Basel, Switzerland) with human Brachyury-encoding pCMV6-ACGFP vector plasmid (OriGene, Rockville, MD) and cultured for 2 days in Advanced-RPMI medium. The transfection efficiency can be validated using flow cytometry to GFP+ cells, and transfection efficiency over 70% is considered successful, generating iNC progenitors. During Step 3, the iNC progenitors, optionally alone or in a mixture with BM-MSCs, were encapsulated in TETRONIC1307-Fibrinogen (TF) hydrogel (in 1-kPa hydrogels at 4° C., 150 μl each, containing 0.1% w/v Irgacure 2959 initiator (Ciba) at a seeding density of 3×10⁶ cells/ml, which are cross-linked under long-wave UV light (365 nm, 4e5 mW/cm² for 8 min), grown in NP-specific medium in hypoxic conditions (2% O₂) for maturation into iNCs in vitro (cultured in culture medium suitable for NP tissue culture, PLOS One. 2013; 8(9):e75548), and harvested for RNA isolation and gene expression analysis or for vibro-sectioning and immunofluorescence staining, if desired. Expression of the main notochordal markers, Brachyury, Noto, Keratin 18, and Keratin 19, can be confirmed as stable in constructs containing iNCs, and slightly elevated from one time point to the next in constructs containing a mixed population of cells. The notochordal markers Br, Keratins 18 and 19, Noto, and Gal3 were expressed in the construct containing only iNCs as well as in the construct containing both iNCs and MSCs (FIG. 3C). After 8 weeks in a 3D culture in hypoxic conditions, MSC-containing constructs showed positive expression of Basp1, CD24, Keratin 8, CTFG, and FOXF1, suggesting a “nucleus pulposus” cell phenotype.

Different cell delivery and microencapsulation techniques will be explored: (1) freshly differentiated cells microencapsulated in synthetic Fibrinogen-F127 (FF) microgel spheres using a microfluidic system, resulting in iNC-microgels, (2) iNC-loaded microgels cultured for 21 days in NP-like conditions to allow the formation of iNC-microtissue through matrix deposition, (3) injection of iNCs into disc-shaped mold filled with FF hydrogel, resulting in iNC in bulk hydrogel. All 3 groups will be tested with different cell densities (0.5×10⁶/ml, 1×10⁶/ml and 2.5×10⁶/ml) to tune for the cell concentration that will promote differentiation and matrix deposition but will also allow sufficient nutrients exchange.

To prepare FF, fibrinogen was first conjugated to PLURONIC®F127 to create a biosynthetic precursor with tunable physicochemical properties based on the relationship between the two constituents; and then a hydrogel matrix could be formed from the fibrinogen-F127 adducts by (1) temperature increase to about body temperature (37° C.) for reversible gelation, or (2) free-radical polymerization using light activation (photo-polymerization). For example, PLURONIC®F127 was end-functionalized with acryl groups and reacted with denatured fibrinogen via a Michael-type addition reaction to form a biosynthetic copolymer (unimer): acrylation of PLURONIC®F127 (BASF™), MW=12.6 kDa, could be carried out under argon by reacting diol polymers in a solution of dichloromethane and toluene with acryloyl-chloride and triethylamine (TEA). A conjugation efficiency can be used to quantify the conversion of free thiols on the fibrinogen to thio-ether linked PLURONIC®F127. In this system, the fibrinogen is a natural substrate for tissue remodeling which contains several cell signaling domains, including a protease degradation substrate and cell adhesion motifs; and the PLURONIC®F127 is a synthetic triblock copolymer (PEO99-PPO67-PEO99) that exhibits a reverse thermal gelation (RTG) property above a critical temperature in aqueous solutions. These materials displayed a reversible temperature-induced physical sol-gel transition and an irreversible light-activated chemical cross-linking. The susceptibility of this hydrogel biomaterial to protease degradation and consequent cell-mediated remodeling was controlled by the PLURONIC®F127 constituent. The protein-based material also conveyed inductive signals to cells through bioactive sites on the fibrinogen backbone, as well as through structural properties such as the matrix modulus (Shachaf et al., Biomaterials, Volume 31, Issue 10, April 2010, Pages 2836-2847).

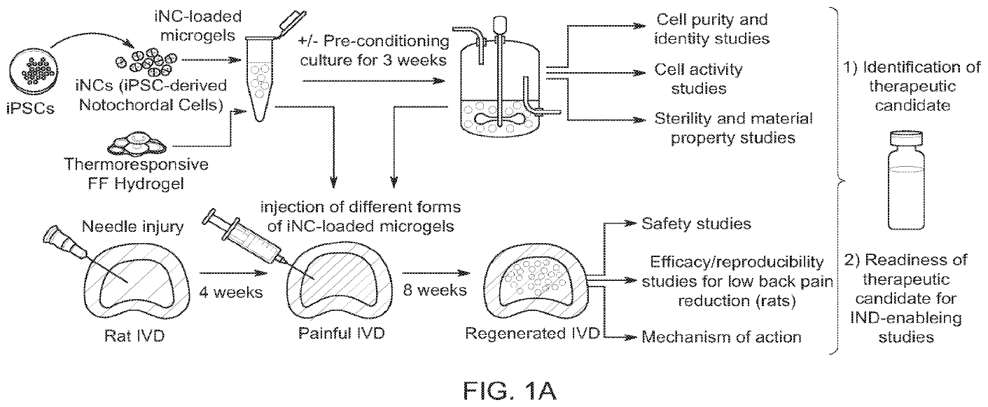

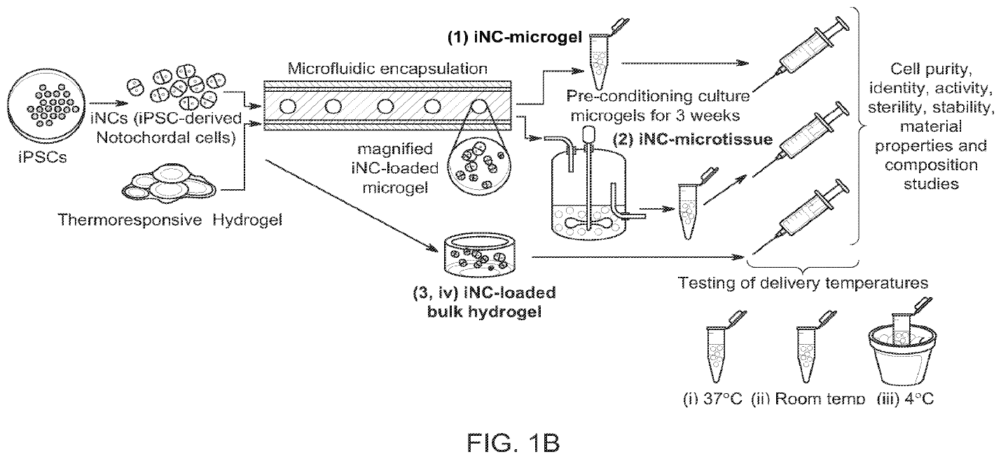

For characterization, the different therapeutic candidates will be passed through a 30G needle and will then undergo the following analyses: cell purity and identity by quantitative iNC cell marker expression analysis, cell viability using CELLGLO™ and PICOGREEN™ assays, sterility tests to ensure absence of viral agents, mycoplasma and endotoxins, evaluation of the material composition consistency and properties of the microgel, microtissue and hydrogel iNC groups using rheometer (FIG. 3D), and optical imaging of cells labeled with fluorescent dyes (FIG. 3F). Hydrogel stability will be tested with Coomassie Brilliant Blue assay under conditions of collagenase-induced enzymatic degradation (FIG. 1B).

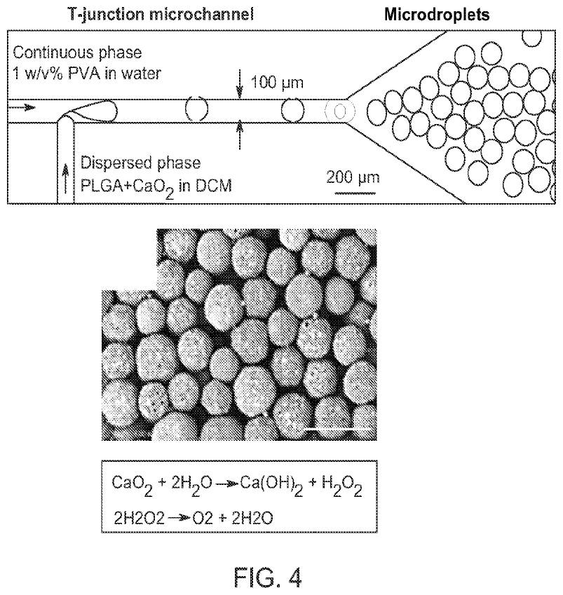

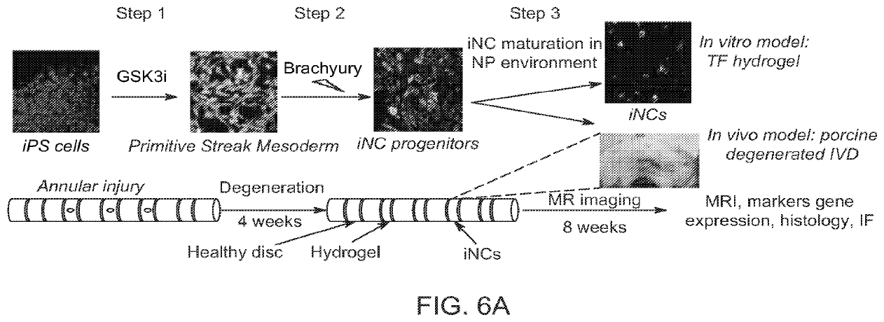

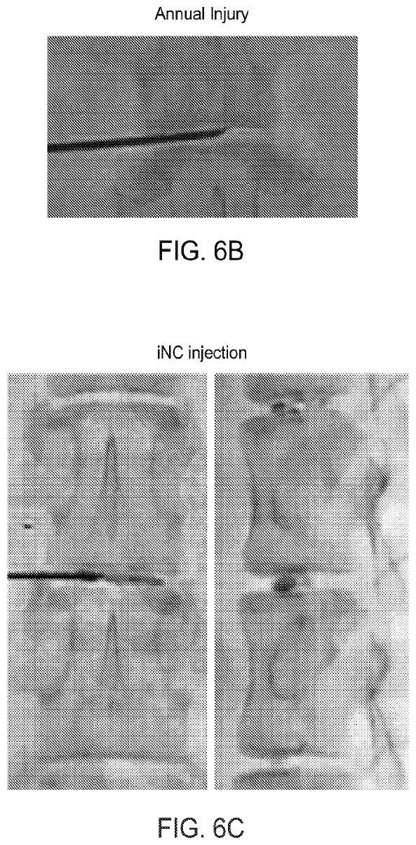

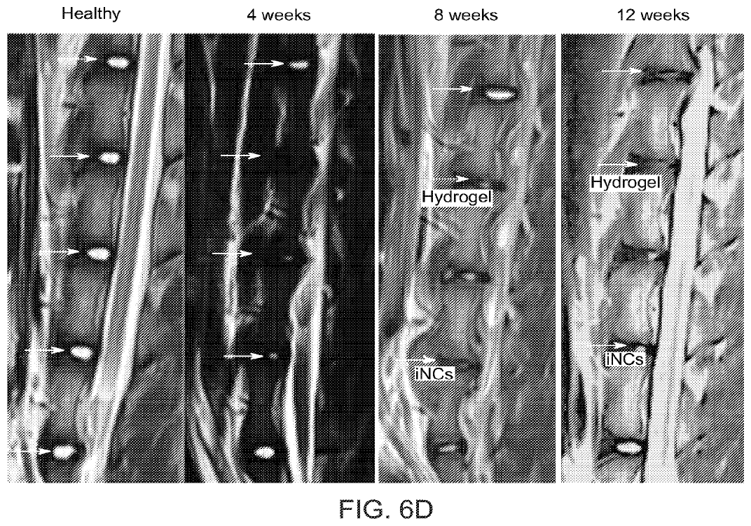

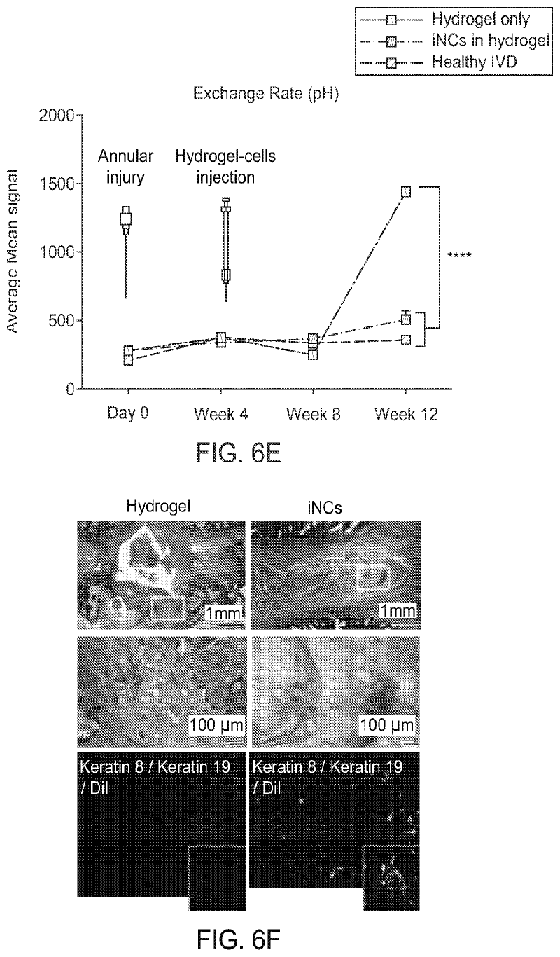

Cell preparation:Human iPSCs obtained from consented patients and generated using a GMP-ready protocol for clinical use were obtained from the Cedars-Sinai Biomanufacturing Center (CBC). The iPSC lines were expanded on animal product-free matrix-coated plates and chemically defined mTeSR™1 media (StemCell Technologies, Inc). Reprograming of iPSCs to iNC was done as described above, also shown in FIG. 6. Briefly, in Step 1, iPSC was treated with 5 μM GSK3i (Millipore) for 3 days. The media was changed daily. In Step 2, the cells were non-virally transfected using the clinical grade closed MaxCyte system with human Brachyury-encoding pCMV6-AC-GFP vector plasmid (OriGene). Cells were cultured for 2 days in A-RPMI media, as reported by Sheyn, D. et al., Stem Cells 26, 1056-64 (2008), lifted, and in Step 3, encapsulated in microgels, shown in FIG. 4. Microgels were either spun down and collected into a Hamilton syringe for injection (Group 1) (FIG. 3); or cultured in hypoxia (2% O₂) and NP media, (Group 2); or non-encapsulated iNCs were mixed with a bulk thermoresponsive FF hydrogel, passed through a needle and collected into cylinder-shaped mold (Group 3).

Microencapsulation: We can formulate FF microgels as depicted in FIG. 3, 4. A reversal thermal gelation approach was used to encapsulate cells in microgels. We used 11.7% (w/v) FF hydrogel in PBS mixed with cells at 10⁷ cells per ml (FIG. 3I); for optimization experiments pre-determined different cell densities (0.5×10⁶/ml, 1×10⁶/ml and 2.5×10⁶/ml) will be used. The FF/cell mixture was loaded into a microfluidic system. Microgels were formed in the microfluidic system and thermo-cured through heat (37° C.).

Microgel stability tests: Microgel or hydrogel stability will be tested with a Coomassie Brilliant Blue assay under conditions of collagenase-induced enzymatic degradation. The tested microgels will contain iNCs at different cell densities (0.5×10⁶/ml, 1×10⁶/ml and 2.5×10⁶/ml) and a microgel control group without cells. The microgels will be cultured and tested for stability at Day 0, 7, 14, 21 and 28.

Cell viability assays: The iNC viability in the microgels at different cell densities (0.5×10⁶/ml, 1×10⁶/ml and 2.5×10⁶/ml) will be tested with CELLGLO™ and PICOGREEN™ assays at Day 3, 7, 14 and 21 to establish viability profile. As positive viability control (100% viability), the same number of 2D cultured iNCs will be used. Cell morphology in the 3D space inside the microgels will be characterized using cryo-electron microscopy, microstructure and porosity of microgels embedded with iNCs at selected cell densities (with highest viability) will be shown by the cryo-EM right after encapsulation and at Day 21 of preconditioning.

Cell identity tests: The iNC identity in the microgels at different cell densities (0.5×10⁶/ml, 1×10⁶/ml and 2.5×10⁶/ml) will be determined by gene expression of NC markers (Br, Keratin 8 and 18, BASP1, SHH, and FoxA2) measured using qRT-PCR and immunostaining. Freshly prepared iNCs will be used as control.

Sterility tests: The sterility of iNC-loaded microgels will be examined by testing the expression of 16S rRNA using RT-qPCR. In addition, standard USP sterility and Mycoplasma tests will be performed according to manufacturer's protocol.

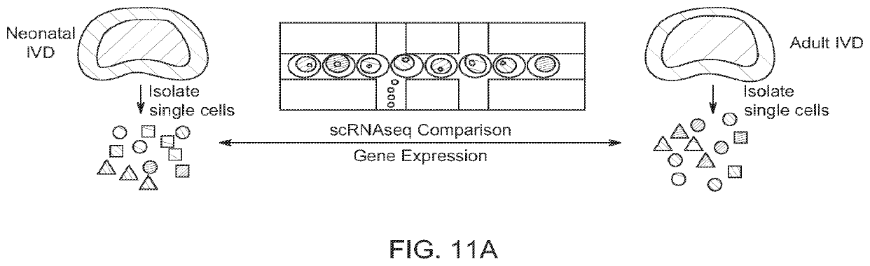

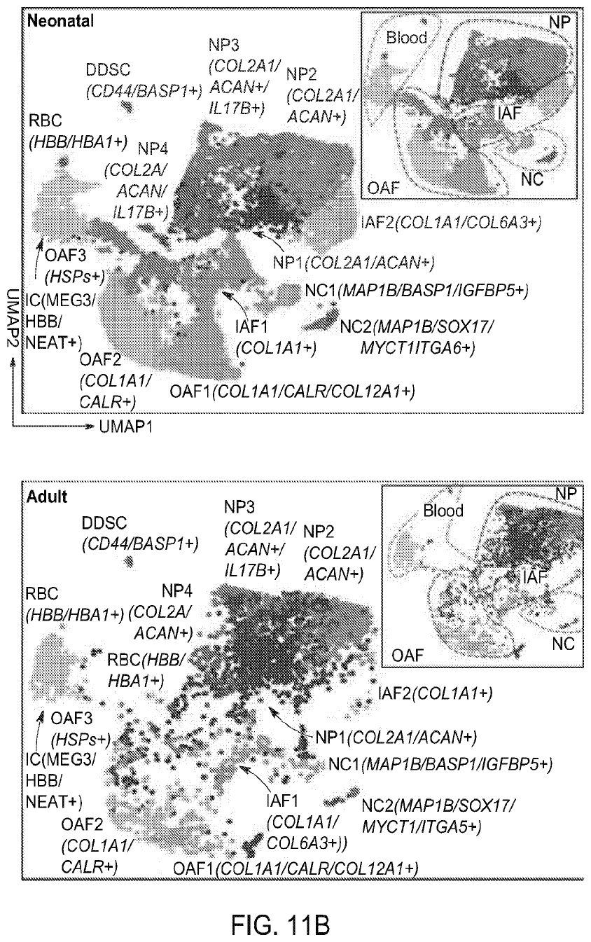

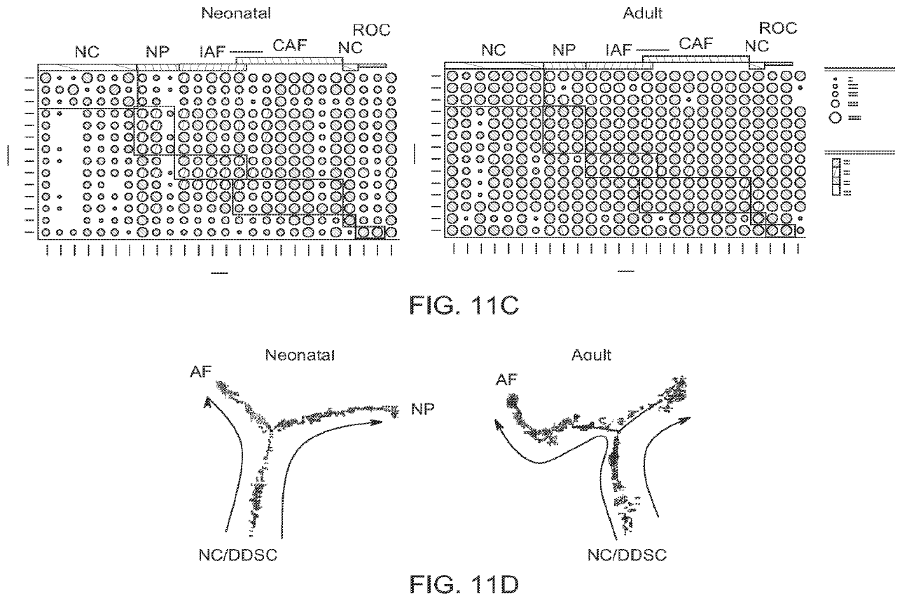

One goal of this study is to develop an injectable, efficient and safe stem cell therapy that can be available to a wide range of patients. Shipment of the therapeutic agent from the biomanufacturing facility to the clinic should be considered. While thermoresponsive hydrogels have the advantage of low toxicity, since no chemical crosslinking or UV irradiation is involved, and high reproducibility compared to UV-based crosslinking, the stability of microgels at different temperatures and times should be considered when developing the therapeutic candidate. To test the stability of microencapsulated iNCs after preparation and during transport from the biomanufacturing facility to the operating room, the following conditions will be applied on all three groups: (i) iNC-microgels/microtissues freshly prepared in 37° C., (ii) iNC-microgels kept in PBS at ambient temperature for 4, 12, 24 and 48 hours to simulate ambient temperature shipment, (iii) iNC in biomaterials kept in PBS at 4° C. for 4, 12, 24 and 48 hours to simulate shipment on ice, (iv) iNCs kept at 4° C. in solution for 4, 12, 24 and 48 hours prior to combination with bulk hydrogel to simulate separate shipment of cells and hydrogel. Tests for viability and characterization will be performed, and viability of 70% will be tolerated as acceptable. Further in vivo studies will be performed within the timeframe that maintains cell identity and at least a 70% viability. Our preliminary data (FIG. 11, 12) demonstrated some microgel formulation and characterization. The mechanical property of the microgels can be adjusted, or alternative materials such as Ge1Ma or PGLA (FIG. 4) can be used, so that the cells may retain and secrete extracellular matrix. UV-activated crosslinkers could also be used during the microencapsulation process to create a more stable hydrogel (than thermoresponsive one without UV-induced crosslinking) for shipping.

### Example 2. Studies to Determine Safety and Efficacy of iNC-Loaded Microgels/Microtissues or iNCs Injected in Bulk Hydrogel in a Rat Model of Disc Degeneration and Discogenic LBP

Immunocompromised (Nude) or Sprague Dawley (SD) rats will undergo spinal disc puncture at 2 consecutive lumber levels, L4-5 and L5-6, using an 18 gauge (18G) needle to induce IVD degeneration and discogenic back pain. Rats will undergo μMRI at week 4 post-disc puncture to visualize successful induction of IVD degeneration. In a second procedure at week 4, the different treatment candidates will be injected into the degenerated IVDs (with a smaller 27G needle) and their regenerative potential monitored for an additional 8 and 16 weeks. Only one treatment will be administered to each rat. Safety data for each therapeutic candidate will be collected through general health observations, complete blood counts with differential (CBC-D) and gross necropsy post-sacrifice. For efficacy, the rats will undergo biobehavioral testing (BBT) for hypersensitivity to mechanical and cold stimuli, motor ability and conditioned place preference, μMRI pre- and post-treatment, and immunohistochemical (IHC) analyses (using markers for iNCs, matrix degradation, inflammation and pain) at end points (week 12 and 20 post injury). Nude and SD rats will be treated in vivo with intradiscal iNC-loaded microgels (e.g., 2.5×10⁶/ml) and compared to saline control. Unless the cell survival is significantly lower in SD rats or signs of immunoreaction due to the xenograft approach are detected, SD rats will be used in further studies to investigate the safety and efficacy of the therapeutic candidates: (1) iNC-microgel; (2) iNC-microtissue; (3) iNC in bulk hydrogel; and controls: (4) microgel only; (5) iNC in saline, and (6) saline.

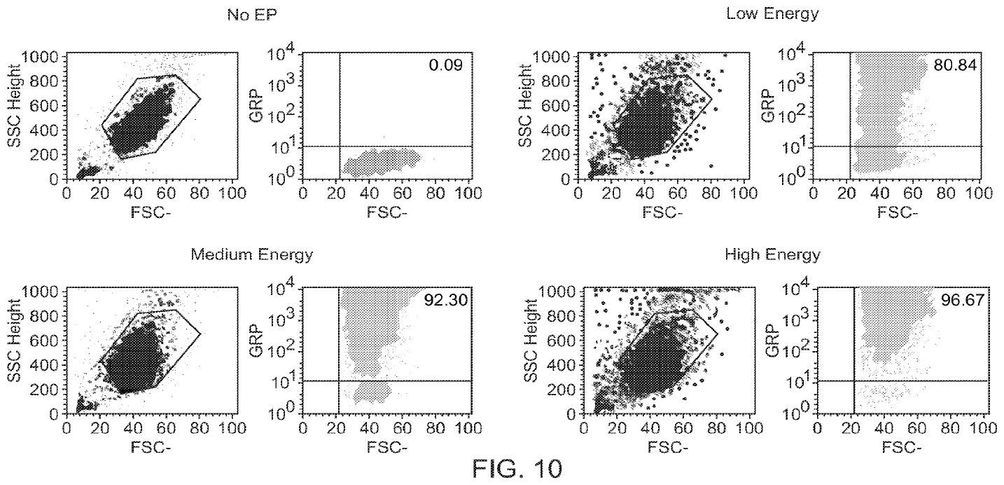

We previously studied the discogenic pain rat model by comparing different needle sizes for induction of IVD degeneration that allow for testing of different therapeutic candidates (Glaeser, J. D. et al., JOR Spine 3, e1092 (2020)). The iNCs' effect on disc degeneration was studied in a large animal model (Sheyn, D. et al., Theranostics 9, 7506-7524 (2019)). However, it is impractical to assess lower back pain (LBP) using biobehavioral tests in large animals (Ison, S. H., Front Vet Sci 3, 108 (2016)). Therefore, we propose to test the iNC therapy in a rats. In each in vivo experiment, safety data for each therapeutic candidate will be collected through general health observations as well as whole blood testing (complete blood counts with differential, CBC-D). Furthermore, each animal will undergo gross necropsy post-sacrifice. In our previous study in a large animal model, no adverse events associated with xenotransplantation of human iNCs to porcine degenerated IVDs were observed. Also, no inflammation was observed when human NPCs were injected into rat IVDs in our preliminary studies (FIG. 10). The cell viability post transplantation can be quantitatively analyzed using flow cytometry, however, to distinguish between the iNCs and the host rat NPCs, we will use iPSCs labeled with Green Fluorescent Protein (GFP) reporter gene to produce iNC-GFP. Since the cells may be rejected without major inflammatory response, we plan to assess cell survival in immunocompetent Sprague Dawley (SD) rats compared to immunocompromised (Nude) rats post iNC-microgel injections into the lumbar IVDs of these animals. We hypothesize that no differences in cell survival will be observed between the two rat strains and plan to use SD rats in the subsequent experiments.

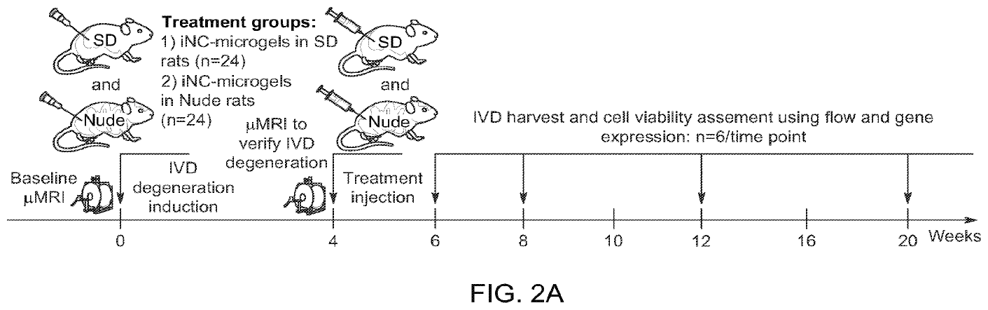

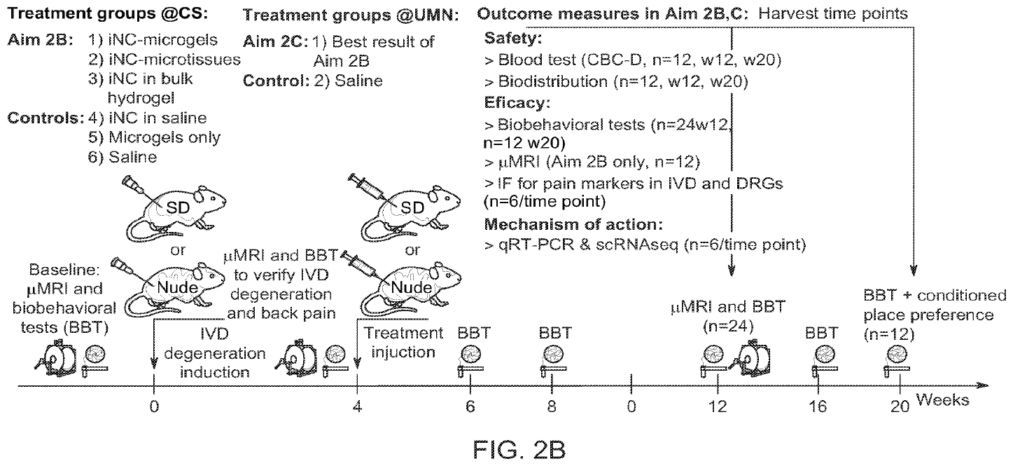

Nude and SD rats will undergo IVD puncture at 2 consecutive lumber levels, L4-5 and L5-6, using a 18G needle to induce IVD degeneration and discogenic LBP. Rats will undergo μMRI at week 4 post-disc puncture to visualize successful induction of IVD degeneration. In a second procedure at week 4, the iNC-GFP will be prepared from iPSCs pre-labeled with GFP reporter gene (Sheyn, D. et al., Mol Pharm 8, 1592-601 (2011)), encapsulated into iNC-microgels with a selected cell density based on Example 1 and will be injected into the degenerated IVDs using a 30G needle in order to minimize the additional damage to the annulus fibrosus. Based on power analysis it will be sufficient to use total of 24 rats per group (12 male and 12 female rats), which will include two treatment groups: 1) iNC-microgels in SD rats; 2) iNC-microgels in Nude rats. Adjacent non-injured discs in each animal will serve as internal “no injury” control. The rats will be euthanized at four time points (2, 4, 8 and 12 weeks, n=6 rats each). The rat spines will be imaged using optical imaging and the fluorescent signal will be quantified (similar to that shown in FIG. 3G) and 4 IVDs per rat will be harvested (2 adjoining pairs of injured+treated and uninjured+untreated). One pair of IVDs in each rat per group (n=6 injured+treated, n=6 uninjured+untreated controls) will be digested for cell isolation and flow cytometry analysis to account for GFP+ cells. The other IVD pair from each animal will have RNA isolated for gene expression analysis of human-specific genes, such as those described in Mizrahi, O. et al., Spine J 13, 803-14 (2013) and Glaeser, J. D. et al., Spine J (2020). Biodistribution studies will be performed to assess leakage of iNCs from the therapeutic site (FIG. 2A).

Rat IVD degeneration and intradiscal injections: Animal experiments will be performed according to the Institutional Animal Care and Use Committee approved protocol. Under inhalation anesthesia and after incision, an anterior transperitoneal approach to the lumbar spine will be utilized. Prior to puncture, a mini C-arm will be used to clearly identify the level of each IVD. Using 18G needle, a disc puncture of 2.0 mm in depth (in the middle of the IVD) will be created in two lumbar levels L3-L4 and L5-L6. The peritoneum, fascia and skin will be closed in layers, and warm fluids and pain medication (0.05 mg/kg buprenorphine, SC) will be administered. Four weeks after the IVD degeneration induction, another surgery will be performed to introduce the cellular treatment. Intradiscal injection of cells was shown to be feasible (FIG. 11F). Cell transplantation into L4-L5 and L5-L6 discs will be performed through a 30-G microinjector (Hamilton) under fluoroscopic guidance.

Cell isolation and flow cytometry: After harvesting, total NP cells will be digested enzymatically (n=6) and the cellular components of the NP will be evaluated using flow cytometry. Donor iNC-GFP cells or host rat NPCs will be identified by fluorescent signal of the GFP reporter gene. Additionally, human notochordal and NP surface marker expression (CD24, Glut1) will be assessed by flow cytometry to identify the implanted cells.

Gene expression analysis to quantify human cells: After harvesting IVDs (n=6), total RNA will be extracted from the NP tissue and the expression of notochordal marker genes (Keratins, −8, −18, −19, Br, Noto, BASP1, SHH, and FoxA2) will be assessed using standard Taqman Gene expression assays (ThermoFisher Scientific).

Biodistribution: Different organs (brain, bone marrow, liver, lungs, heart muscle, skeletal muscle, and spleen) will be biopsied immediately after the animal is euthanized and snap-frozen in liquid nitrogen. Then the tissues will be homogenized, and DNA extracted using a DNA extraction kit (Qiagen). Since the rats will be treated with human iNCs that highly express human Brachyury gene, the DNA samples will be tested for human Br using quantitative PCR and normalized to the 18S housekeeping gene (Sheyn, D. et al., Mol Ther 24, 318-330 (2016)).

Previous studies showed that iNCs maintain the notochordal phenotype in vitro and in vivo and mitigate IVD degeneration. Several groups showed the feasibility of cell injection into healthy and degenerated large animal IVDs in vivo or in IVD explants. Our pilot study shows that iNCs can survive in degenerated IVD and sustain their phenotype for at least 8 weeks. Here, we conceive that encapsulation of iNCs in microgels combined with pre-conditioning culture will increase the cells' efficacy in IVD regeneration and mitigation of LBP symptoms compared to other therapeutic candidates and controls. To evaluate the mechanism of action, single-cell RNA sequencing (scRNA-seq) of the treated and untreated IVDs in combination with IHC will be employed. By examining the gene expression of the transplanted cells and the host cells separately using scRNAseq, we will find out the iNC regenerative capacity to secret matrix and correlate their analgetic effect to pain-related markers expression. Furthermore, we will investigate the response of the host rat NPCs to the treatment, including the reduced secretion of known neurotrophic factors and innervation of the IVD. Investigation of the safety and efficacy of the different therapeutic candidates at a density of 1×10⁶/ml and controls: (1) iNC-microgel (2) iNC-microtissue; (3) iNC in bulk hydrogel; (4) microgel only; (5) iNC in saline, and (6) saline control. Rat IVD degeneration will be induced and treated as described in detail above. For efficacy evaluation, the rats will undergo biobehavioral testing for hypersensitivity, motor ability (rotarod and open field), μMRI pre- and post-treatment, and immunohistochemical (IHC) analyses (using markers for iNCs, differentiation, matrix degradation, inflammation and innervation) at the study end. For data analysis, group effects and, if applicable, temporal effects will be evaluated. To get insights into the mechanism of action of the different delivery systems, 3 out of 8 treated rat spines will be used for histology and the treated IVDs from the remaining 6 spines will be used for scRNAseq analysis of cell fate, differentiation state and pain-related mechanisms (FIG. 2B).

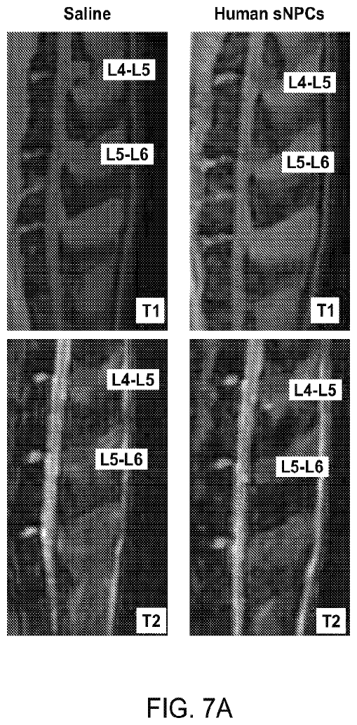

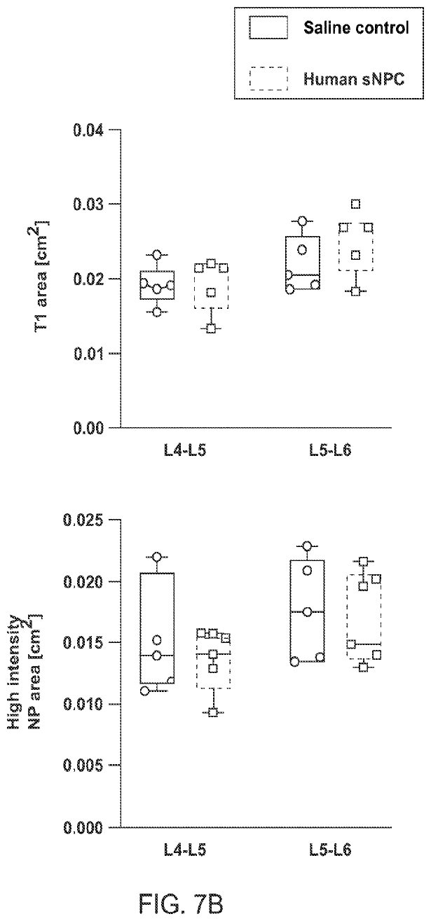

NP matrix imaging using μMRI: To visualize the IVD structure and hydration levels, μMRI imaging will be employed (Bruker BioSpec 9.4T), as described in our preliminary studies (FIG. 7). Each rat will be scanned at baseline, and at 4 and 12 weeks post-puncture (equal to treatment injection and 8 weeks post-injection timepoints). Briefly, anesthetized rats will be placed on the examining bed in prone position. To ensure the optimal angle for sagittal slice scanning, a series of axial, coronal and sagittal pilot proton density (T1) scans (TR: 50 ms, TE: 1.7 ms) will be performed. After obtaining satisfactory sagittal midsection proton density scans for outlining the disc location and size, sagittal proton density scans (TR: 50 ms, TE: 1.7 ms) and T2-weighted scans (TR: 5000 ms, TE: 30 ms) with exact same imaging geometries will be performed. The level of disc hydration will be quantitatively measured using MIPAV computer imaging software (Medical Image Processing, Analysis, and Visualization, NIH). Utilizing the iliac crest as the anatomical landmark for each scan, regions of interest (ROIs) of IVD L4-5 and L5-6 will be manually contoured by 2 independent researchers that are blinded to the conditions for measurements of changes in the disc area (T1-weighted) and high signal area values of the NP (T2-weighted).

Biobehavioral tests: All behavioral testing will be conducted by a treatment-blind experimenter between 3:00 pm and 7:00 pm. To assess the effect of IVD degeneration and the treatments on pain measures, biobehavioral tests will be conducted according to FIG. 2A, as performed in our preliminary (FIG. 10).

von Frey: an electronic von Frey (www.iitcinc.com) device will be used to assess mechanical/tactile allodynia. The animals will be placed in a Plexiglas testing chamber (22 cm×22 cm) with a grid mesh floor. After a 15 min habituation period, a mechanical stimulus will be delivered by applying a von Frey hair alternately under the plantar surface of the left and right hind-paws. The force necessary to produce paw withdrawal or nocifensive behavior will be recorded.

Randall-Selitto test: the Ugo Basile Analgesy-Meter (www.ugobasile.com) will be used to measure mechanical hyperalgesia. The experimenter will gently restrain the rat in one hand for testing on the paw pinch apparatus and with the other hand guide the hind paw to be tested on the plinth under the cone-shaped pusher. A weight operated by the experimenter pressing a pedal-switch will exert a force at a constant rate of 16 grams per second. When the rat will elicit paw withdrawal or show nocifensive behavior, the experimenter will release the pedal and record the applied force. Three measures for each paw will be collected then averaged. For both the Randall-Selitto and von Frey testing the first paw to be assessed will be randomly selected to avoid anticipation by the animal. Paw withdrawal thresholds will be determined for left and right. For data evaluation, withdrawal thresholds from left and right will be averaged.

Cold sensitivity assay spontaneous nociceptive behaviors are monitored using video recording and slow-motion post-analysis for 1 min after a drop of acetone (25 μl) is applied to the plantar surface of the hind-paw with the aid of a blunt needle attached to a 1 ml syringe. The total duration of paw withdrawal, defined as the total time of flinching, licking or biting, is measured with a stopwatch. The total time of response is plotted as performed in our preliminary studies (FIG. 10C, 11A).

Grip test: sensitivity to axial stretch will be assessed using the grip force assay. In this apparatus, wire mesh grip force bars are connected to force gauges. During testing, each animal is held at the base of its tail and gently passed over the wire mesh grids. The strain gauges convert forelimb grip force at the time of release to a digital readout.

The Rotarod assay will be included as a measure of motor capacity. Movement-evoked pain or impairment will be assessed during ambulation on a Rotarod (San Diego Instruments). The rats will be placed on the rotating rod for a 210-s trial repeated three times at 30 min interval. For each trial, the rod will be set at a start speed of 3 rpm that remained constant for 30 s, then the rod gradually accelerated from 3 rpm to 30 rpm over a 3 min period. The latency to fall off the rod will be averaged across the trials (FIG. 2B).

The open field assay is included as a measure of rearing, motor capacity and anxiety. After 30 minutes of habituation to the testing room, locomotion will be assessed over 5 min in a 100 cm×100 cm×50 cm plexiglas open field with grey floors and transparent sides. Animals are videotaped and activity is quantified later by a blinded observer using AnyMaze software. Measures will include rearing, total distance, and time spent in the center of the open field.

Analysis of harvested of IVDs and DRGs: At week 12, half of the rats (n=12/group), will be euthanized, the IVDs at spinal level L3-L4 (as control), L4-L5, L5-L6 and the corresponding DRGs will be harvested for gene expression analysis. Briefly, RNA will be isolated using the Qiagen RNeasy mini kit (Qiagen) and transcribed to cDNA using the High-Capacity cDNA Reverse Transcription Kit (Applied Biosystems). Using TaqMan gene expression assays, analysis of the following genes will be performed: 1) inflammation-related genes, NFKBAI, TNFα, IL1β, IL6, IL8, IL17, and IFNγ, 2) pain-related genes CGRP, ATF3, NGF and BDNF, and 3) IVD degeneration markers CNN2, MMP3, AGC, Col I and II, and 4) human notochordal markers. Rats for histology (n=6/group) will be anesthetized and transcardially perfused with saline and 4% PFA. IVD, DRG and spinal cord tissues will be collected and fixed for an additional two hours in 4% PFA. The DRGs will be transferred to 30% sucrose for 48 h for cryoprotection before sectioning at 35 μm. Spines will be decalcified and embedded in paraffin, sections will be analyzed for morphological changes using standard H&E, and Picrosirius Red/Alcian Blue stains. To investigate the degeneration and inflammation state of the IVDs staining will be performed against TNFα, IL-1β, IL-6, as well as NP degeneration markers (CNN2, MMP3). To examine the effect of treatment on pathological disc innervation, IVDs will be stained with pain markers PGP9.5 and CGRP, and performed in our preliminary results. To determine the effect of treatment on sensory neuroplasticity, immunohistochemistry will be performed on DRG for CGRP and NPY and in spinal cord for CGRP, NPY, GFAP for astrocytes and Iba1 for microglia.

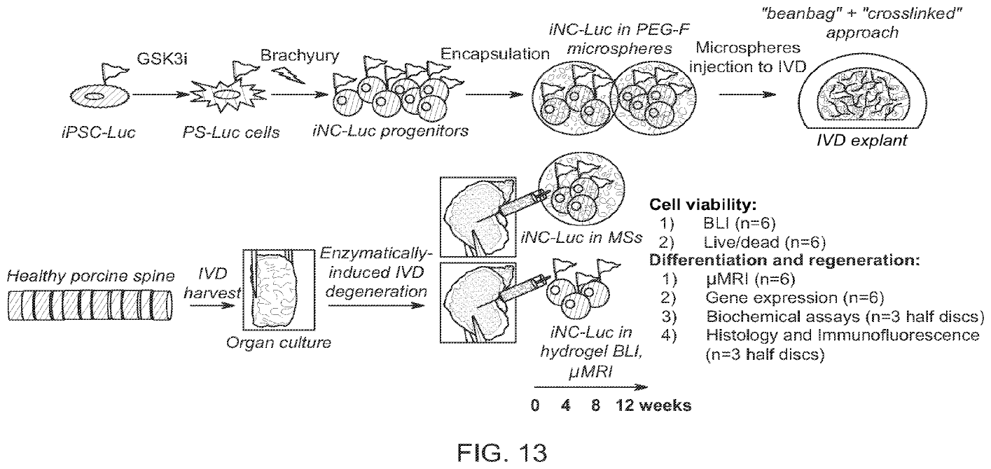

Single-cell RNA sequencing (scRNAseq): For cell identity purposes, treated IVDs will be harvested, enzymatically digested, cells isolated. For each sample, Chromium Single Cell 3′ v3 libraries with ˜3,000 cells will be prepared on a Chromium Controller with chips and reagents from Single Cell Gene Expression v3 kits following the manufacturer's protocols (10× Genomics). Then, the libraries will be sequenced using paired-end sequencing (28 bp Read 1 libraries, and 91 bp Read 2) with a single sample index (8 bp) on an Illumina NovaSeq. Samples will be sequenced to a depth of >50,000 raw reads per cell, with raw sequencing data analyzed and visualized with pre-release versions of Cell Ranger 3.0.0 and Loupe Cell Browser 3.0.0. Upon completion of all scRNAseq runs, only single cell libraries that pass quality control filters will be aggregated across all experimental batches and analyzed together. We will compare their cell subsets and cell types across different samples. We will further compare the expressions of NP, AF or NC-relevant genes at single cell resolution similarly to shown in FIG. 13.

Reproducing the results of the most promising therapeutic candidate compared to untreated sham control will ensure that we are taking a viable candidate through the translational pathway and will facilitate the creation of the Target Product Profile (TPP). Expansion of the in vivo testing battery top include lateral flexion-related fatigue and conditioned place preference will further strengthen the supporting data and extend the results to spontaneous pain. Finally, expanding the rat model to a second test site will support the higher volume work required for future IND-enabling studies. To test for reproducibility, the most promising therapeutic candidate identified in Example 2 above will be shipped using the condition described in Example 1 and tested at a selected dose and compared to sham control (n=12). The rat IVDs will be degenerated, treated and monitored. The outcome measures will include BBTs, and in addition, FlexMaze will be performed. At endpoint IVD and DRG histological analysis will be performed at Week 20. An additional biobehavioral test may be performed: the FlexMaze assay, as a measure of sensitivity to spinal flexion. Animals are allowed to explore a plexiglass maze with a series of sharp left and right angles. The total amount of exploration as a function of time will be measured. A decrease in time suggests the development of hypersensitivity to lateral movement (FIG. 11). Conditioned place preference will be performed using intrathecal clonidine to unmask spontaneous pain by preference for the analgesia-paired chamber as supported by preliminary data FIG. 11. According to our results (FIG. 6), and due to the fact the IVD is considered immunoprivileged, we believe that the iNCs will survive and retain their phenotype in the IVDs of both the SD and Nude rats. In case the survival of cells will be lower in SD rats than in Nude rats, we will use Nude rats for Example 2. Moreover, we believe that iNCs will attenuate disc degeneration after annular injury and will reduce the pain measurements (FIG. 1A). All testing will be performed at the two-sided significance level of α≤0.05. Analysis will be performed with one- or two-way ANOVA as appropriate, with Bonferroni correction for multiple testing. Repeated measures (over time and/or across matched sets of IVDs from each rat) will be tested with mixed model regression. Analysis will be performed with Graph Pad Prism v9.1 or SAS v.9.4 software packages. Experimental sample sizes were computed based on power analysis from preliminary data. For in vitro studies in Example 1, all experiments will be performed with at least 3 biological replicates, with 3 experimental replicates to confirm findings. For Example 2, n=6 IVDs per timepoint to assess cell survival rates and over time will have 90% power to detect an effect size of 0.5 between mouse strains (SD vs Nude) assuming group variation is 10% or less; and based on pilot Von Frey data comparing rats with injured vs. non-injured discs 8 weeks after disc injury in a pilot experiment with n=7 per group (FIG. 10C) assuming a standard deviation of 2.5 and a correlation of 0.5 between repeated measures, n=12 per timepoint will have >90% power to detect expected differences between groups of 2.0 units. (Power computed with PASS software 2020.) Sample sizes will be a minimum of n=6/group for μMRI studies, n=6 IVDs for scRNAseq, and n=6 IVDs for histological analysis and staining and n=12 for biobehavioral endpoints.

Overall, our new approach of intradiscal injection of iPSC-derived notochordal cells (iNCs) is a first-in-class treatment for painful IVD degeneration. This approach of embedding iNCs into a microgel/microtissue delivery system that promotes cell function and survival, has the potential to rejuvenate IVDs, attenuate disc degeneration and prevent discogenic pain in individuals suffering from intervertebral disc disease. Development of an allogenic stem cell therapy will allow for an off-the-shelf treatment accessible to different population groups suffering from painful IVD degeneration. The proposed therapy could also be adapted to use autologous patient-derived materials.

### Example 3. Preliminary Studies

The role of NCs in the IVD. The NP is formed from the embryonic notochord as it segments during fetal development; the surrounding annulus fibrosus (AF) is formed from the sclerotome/mesoderm. At birth, the NP is populated by morphologically distinct, large vacuolated NCs. In some vertebrates these NCs persist throughout most of adult life, whereas in other species, including humans, these NCs gradually disappear during maturation, eventually becoming undetectable and are replaced by a population of smaller round cells—NP cells—believed to differentiate from NCs. The change in cell population correlates with the initiation of degenerative changes within the disc. Animals in which NCs remain throughout the majority of their lifespan, including rabbits, rats and mice, maintain a more hydrated, proteoglycan-rich NP matrix than human adults. Supporting this theory, NCs are more metabolically active and produce more proteoglycans than NP cells. In vitro experiments with human and bovine NP cells encapsulated in 3D hydrogels indicate that NC cells could also act as stimulators, inducing the synthesis of proteoglycans by the NP cells. Furthermore, notochordal cells protect NP cells from apoptosis during IVD degeneration. Based on these findings, a stem cell therapy using NC cells may be more efficient in proteoglycan-rich matrix induction than NP cell therapy and may have protective effect on the host NP cells from the pathological environment.

iPSCs as a cell source for IVD cell therapy. Given the evidence above, there is a shortage in cells that can repopulate a degenerated IVD. Thus, a potential solution is to mimic the process IVD formation that occurs during embryogenesis in iPSCs. Unlike embryonic stem cells (ESCs), iPSCs are controversy-free and can be generated from almost any somatic cell using integration-free methods. Others have shown the feasibility of iPSC differentiation towards NC-like cells using non-defined NP tissue matrix (Liu, Y., PLOS One 9, e100885 (2014)): direct contact between hiPSCs and NP matrix can promote the differentiation yield, whilst both the contact and non-contact cultures can generate functional NC-like cells; and a culture medium containing a cocktail of growth factors (FGF, EGF, VEGF and IGF-1) also supported the notochordal differentiation in the presence of NP matrix. We have recently developed iPSC differentiation to iNC using a stepwise approach (Sheyn, D. et al., Theranostics 9, 7506-7524 (2019)), detailed in Example 1.

Differentiation of iPSCs to iNCs and testing efficacy in a large animal model. During the third week of embryonic development, after the gastrulation and formation of the three layers, the mesoderm undergoes subdivision into axial, paraxial, intermediate and lateral plate mesoderms. This subdivision occurs by formation of primitive streak (PS) and the axial mesoderm precursors ingression via the most anterior aspect of the PS. Thus, PS cells can be identified as precursors of the notochord. The activation of the Wnt pathway by blocking glycogen synthase kinase-3 (GSK3) using chemical inhibitors was previously shown to induce ESCs and iPSCs differentiation towards PS cells. Overexpression of Brachyury transcription factor was demonstrated in our previous study to differentiate PS cells to iNC (FIG. 6A). In response to injection of human iNCs into degenerated porcine IVD in bulk thermoresponsive hydrogel (Geltrex™, FIG. 6B-6D) compared to hydrogel only or bone marrow (BM)-derived MSCs, increased protection against IVD degeneration associated pH changes (imaged with MRI) and morphological changes characteristic for IVD degeneration in combination with iNC cell survival and retention of the iNC phenotype were detected (FIG. 6D-6F). However, there was no complete regeneration and new matrix formation. The porcine disc degeneration model has the advantage of a similar size to humans, similar mechanisms for intradiscal therapeutic delivery (posterolateral spinal access) and it allows for the use of similar imaging techniques (MRI) to assess therapy success. Downside of this animal model is the limiting ability to assess pain using biobehavioral tests. Therefore, we propose to use the rat model in this stage and, if successful, to incorporate the porcine model for the IND-enabling studies later.

Rat model for discogenic pain. Degenerative changes in the IVD are often associated with nerve ingrowth and hyper-innervation. While MRI provides detailed images of the IVD, it fails to clearly differentiate between a painful and a non-painful disc. Commonly used IVD degeneration animal models include mouse, rat and pig. The advantages of the rat model are its relatively small size, the usability in large cohort studies, its relatively large discs compared to the mouse allowing for injection of therapeutic agents, and the support of biobehavioral testing—an important component in studying discogenic pain. We developed a method to reliably control the degree of lumbar IVD degeneration and associated pain responses by the size of the needle used for puncture in Sprague Dawley (SD) rat: Fluoroscopy-guided needle injury was performed in lumbar discs (L4-5, L5-6) and followed up for 8 weeks. While IVD puncture with a 21G needle clearly demonstrated signs of moderate disc degeneration, injury with 18-gauge (G) needle induced severe disc degeneration and behavioral signs of discogenic pain. Evidence was provided by μMRI analysis, correlation analysis between needle diameter and μMRI results and histology. In a similar model in mouse, pathological innervation was observed in injured discs.

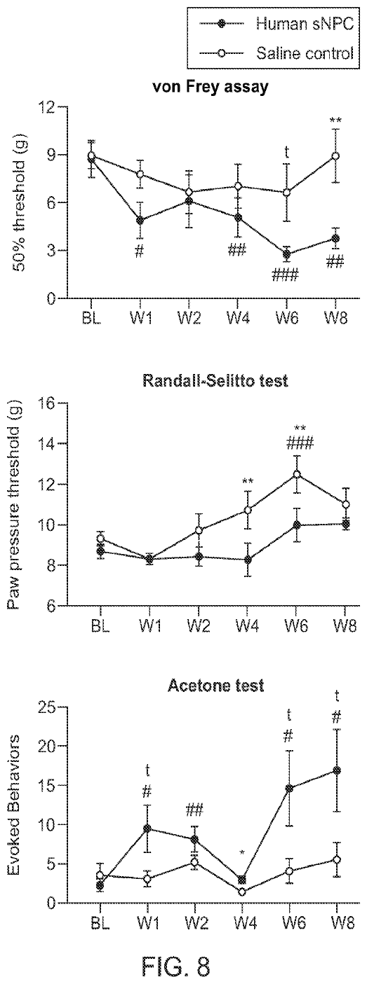

Human stressed NPCs induce discogenic pain in vivo. In parallel studies we explored the effect of injecting stressed human NPCs (sNPCs) on IVD degeneration and discogenic pain in rats. In brief, human NPCs were exposed to cell stressors mimicking the degenerating IVD (hypoxia, low glucose, low pH, and IL-1β). 10 μl of saline with and without 4×10⁴ stressed NPCs were injected using a 31G needle into two levels of the rat IVDs, followed by μMRI (FIG. 7) and biobehavioral tests (BBTs) (FIG. 8). μMRI of the rat IVDs at week 6 showed no signs of disc degeneration in any group (FIG. 7). BBTs (von Frey, cold sensitivity and Randal-Sellito) resulted in increased sensitivity in rats injected with stressed NPC (but not saline) as early as week 1 (von Frey) and up to week 8 (all three tests, FIG. 8). These findings support the feasibility of the current proposal by demonstrating signs of LBP detected with BBTs after injection of sNPCs.

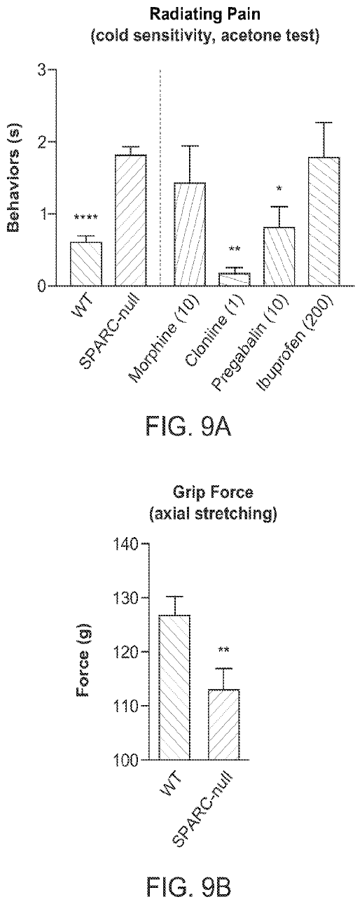

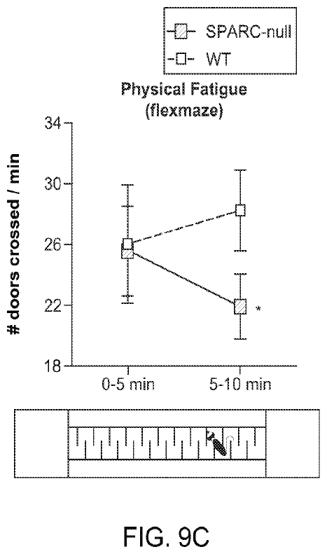

Mouse biobehavioral studies in LBP. A primary goal of this project is to move iNC-based intradiscal therapy towards clinical development for the treatment of chronic LBP. In the current project, we will extend the battery of BBTs beyond mechanical and cold sensitivity tests (FIG. 9, panel A) to include the grip force assay for axial discomfort (FIG. 9, panel B), the open field and rotarod assays for motor ability and we will employ our FlexMaze assay for rats to measure use-related discomfort due to lateral flexion (FIG. 9, panel C). The progressive slowing of exploration speed is reminiscent of increased fatigue experienced by chronic pain patients.

Nonviral transfection: To optimize the nonviral transfection, the MaxCyte system was employed using 5×10⁶ cell/20 μg GFP plasmid and MaxCyte EP buffer according to manufacturer's protocol. Different energy protocols were tested to achieve the best efficiency/viability balance (FIG. 10).

Fibrinogen-F127 microgel cell delivery system: We prepared cell-encapsulating FF microgels from FF precursors using reversal thermal encapsulation. We first prepared no-cell microgels, as shown in FIG. 3A. The gelatinized microgel gradually settles down to the bottom of a 50 ml tube. We then encapsulated NPC in FF microgel (FIG. 3B). The FF gel precursors are liquid in 4° C. (FIG. 3C left) but turned into solid gels at 37° C. (FIG. 3C right). The rheometer test shows the thermal gelation temperature at around 21° C. (FIG. 3D). The NPC encapsulated in FF microgels maintained >80% recovery after 7 days of preconditioning compared to day 0. (FIG. 3F) Spreading morphology of NPC was observed after 21 days of preconditioning using confocal microscopy. Fluorescent signal from the disc can be imaged and quantified using optical imaging (FIG. 3G). That is, we first prepared no-cell microgel as shown in FIG. 3A. The gelatinized microgel gradually settles down to the bottom of a 50 ml tube. The FF gel precursors are liquid in 4° C. but turned solid at 37° C. (FIG. 3C). Rheometer tests show the thermal gelation temperature at around 21° C. (FIG. 3C). We then encapsulated cells in FF microgel, and spreading morphology of NPCS was observed after 13 days of culture in vitro (FIG. 3D). Injectability tests of the microgel indicated a 18G needle to be ideal for gel injection into porcine disc (FIG. 3H).

Fabrication of oxygen-releasing microparticles (MPs): We made a microfluidic system to generate poly (lactic-co-glycolic acid) (PLGA) MPs (FIG. 4). We showed that 3% (w/v) of calcium peroxide (CPO)-loaded Ge1MA hydrogels possessed no cell toxicity, while improving cell viability in hypoxic conditions (Alemdar, N. et al., ACS Biomaterials Science & Engineering 3, 1964-1971 (2017)). Having had experience in MPs' generation, we successfully established a protocol for CPO encapsulation (up to 5% (w/v)) in PLGA MPs using the microfluidic system. The MPs of size ˜50 μm were generated. Scanning electron microscopic (SEM) images revealed that CPO-loaded PLGA MPs showed a rough surface due to the spontaneous oxygen-releasing from the encapsulated CPO (FIG. 4). In contrast, a smooth surface was observed for pristine PLGA MPs.

Single-cell RNA sequencing (scRNA-seq). We conducted scRNA-seq of neonatal IVD tissues harvested from surgical discards of a human infant (FIG. 11, panel A). Samples were sequenced to a depth of >50,000 raw reads per cell, with raw sequencing data analyzed and visualized with Seurat package in R. After filtering out low-quality and abnormal data, we conducted cell Uniform Manifold Approximation and Projection (UMAP) which identified 14 clusters as shown in FIG. 11, panel B. We have specifically plotted the projection of expressed gene markers that are relevant to NP cells and NC cells and AF cells (FIG. 11, panel B). We described the averaged gene expression level of each cell subset and the percent of cells expressed in a dot plot as shown in FIG. 13C. The cell type of each subset was identified based on the gene expression projection and dot plot. We also created pseudo-time trajectories for neonatal and adult IVD in FIG. 13D, which identified the different stages of development in neonatal and adult IVD.

Summary of preliminary data: Our data show the feasibility of iPSC differentiation to iNC, and their survival and retention of the iNC phenotype in vivo (FIG. 6). We have established a model of IVD degeneration with measurable outcomes of discogenic pain in rats (FIGS. 7, 8). We have shown the ability to image and quantitively analyze disc degeneration using μMRI (FIG. 7), assess the innervation of the IVD following degeneration and demonstrated the expertise to perform the in vivo biobehavioral assays (FIGS. 8, 9). Our preliminary results demonstrate our ability to perform efficient cell transfection using scalable and GMP-ready the MaxCyte system (FIG. 10), generate microgel encapsulating iNCs and to deliver them to the site of IVD degeneration (FIG. 3, 4). We provide evidence of our capability to perform single-cell RNA sequencing and downstream analysis including UMAP, clustering and lineage tracing (FIG. 11). Taken together, studies described in Examples 1 and 2 are supported by our preliminary data.

### Example 4. Vertebrate Animals and Biological and Chemical Reagents/Materials

Rats (Sprague Dawley; Nude) will be obtained from Charles River (mean weight 200 g; ˜7-9 weeks old).

Anesthesia and Pre-Op Procedure: Animals will be prepared following Pre-Surgical Preparation. First, animals will be anesthetized (Isoflurane Anesthesia). Ophthalmic ointment will be placed in the animal's eyes to prevent corneal drying. Animals will be given thermal support (temperature-regulated heating pad or heat lamp placed approximately 18 inches from the animal or cage) for the duration of the anesthetic episode. After anesthesia induction and before the start of the surgical procedures, carprofen (5 mg/kg) and buprenorphine (0.1 mg/kg) will be injected subcutaneously.

Hair (if any) on the surgical site will be clipped using electric clippers. The surgical site will be aseptically prepped by thoroughly disinfecting with betadine or chlorhexidine followed by alcohol in alternating wipes. Sterile drapes will be placed as needed to ensure a sterile field surrounding the incision site and for an area to place sterile instruments. If the sterile field or surgical site must be digitally manipulated, sterile gloves will be used. After anesthesia and prior to surgery, rat will be marked by following Ear Notching guideline. Animal is moved to the operating table and placed in dorsal recumbency.

Surgical Procedure: An abdominal straight incision (˜7 cm) is made with a sterile surgical scissors. The abdominal incision is extended through the linea alba into the abdominal cavity. Note that incisions with surgical scissors, as opposed to a scalpel, reduces bleeding and the risk of damage to the underlying tissues. The intestines are deflected to the rat's right to expose the abdominal aorta and the left kidney. Anatomical landmarks are then palpated to determine the spinal region to be exposed in upper caudal vertebrae. The anterior edges of the spinal column are isolated from connective tissue and muscle. Blunt dissection, rather than cutting, reduces bleeding, decreases risk of hematoma (extravasation of blood outside the blood vessels), and infection, and facilitates healing. A cotton swab saturated with dilute hydrogen peroxide (3%) will be used to remove blood and residual tissue from the spine. Prior to puncture, a mini C-arm is used to clearly identify the level of each intervertebral disc. Using a sterile needle, a disc puncture of 1.5-2.0 mm in depth (to middle of intervertebral disc) will be created inside the intervertebral disc. For each level, one sterile needle will be used for puncture. After the puncture is completed, the tendon and tissues are placed back. The body wall layer (Linea Alba) is closed using vicryl synthetic absorbable surgical suture in a continuous pattern. The subcutaneous tissue layer is closed using monofilament synthetic absorbable surgical suture in a continuous pattern. The skin is closed using monofilament nylon non-absorbable suture in a simple interrupted pattern.

Post-Op Care: After surgery, animal will be treated by following Post-Surgical Care-Major Survival Surgery guideline. (Note: only warm normal saline will be given to the animal. Lactated Ringers Solution will not be administered). Due to the immune-compromised nature of diabetic rats, SC antibiotic will be administered 3 days post-op.

Post-Op Procedures: In-vivo small animal MR imaging will be performed on all animals under approved IACUC Protocol: small animal MR Imaging for Rat by imaging core staff. Animal will be anesthetized using inhaled anesthesia.

In-vivo Behavioral testing will be performed on all animals under approved Core Protocol: Behavioral Testing in Rats. The following tests will be performed by the Biobehavioral core staff on the same day: Cold sensitivity, von Frey Hair Stimulation, and Randall-Selitto.

The IVD degeneration model in rat has several similarities to its human counterpart and therefore it is a well-established animal model for the evaluation of new approaches for the treatment and regeneration of disc injuries including the treatment of nucleus pulposus degeneration following needle puncture.

After anesthesia induction and before the start of the surgical procedures, carprofen (5 mg/kg) and buprenorphine (0.1 mg/kg) will be injected subcutaneously. After surgery, rats will be transported to vivarium. To limit discomfort, distress and pain Cedars-Sinai comparative medicine clinical staff and the study staff will perform the following procedures: Animals are monitored once daily during the first three days of post-surgery weekdays, weekends/holidays (prior to 10 AM). Animals will be given analgesia the day after surgery by study staff. During these 3 days monitoring period, water-soaked chow in petri dishes will be placed by study staff on the cage floor to ensure the animal's ability to get adequate food and water. Research study staff observe the animals in their cages and when handling for any signs of distress such as (but not limited to) continual rubbing of the wound area, vocalizations, teeth grinding, or lack of cleaning, feeding and drinking. Long-acting buprenorphine 0.1 mg/kg (IM) will be administered to the animal after surgery for post-operative pain relief and this will be repeated as necessary by Comparative Medicine clinical staff and attending veterinarians for up to 72 hours post-operatively as required. Animals displaying other signs of pain or distress such as reduced activity, hunched, ruffled (piloerection), and/or thin (body condition score 2-3 out of 5) will be reported to Comparative Medicine clinical staff and treated if possible.

At the end of each time point or by recommendation of attending veterinarians due to complications, the animals are euthanized following method that is consistent with American Veterinary Medical Association (AVMA) guidelines.

Brachyury plasmid purchased from Origin will be sequenced at the beginning of the project and tested using qPCR and/or restriction enzyme digestion followed by gel electrophoresis after every expansion cycle.

Cell culture media and all supplements (including plate coating materials), as well as disposable laboratory materials, will be purchased from certified and known vendors (Thermo Fisher Scientific, StemCell Technologies, Sigma-Aldrich).

Cell viability assays will be done using the quantitative CellTiter-Glo 3D assay kit (Promega Madison, WI) per manufacturer's protocol, as previously done in preliminary studies.

Cell toxicity levels will be measured using LDH assay (ab65393, Abcam) according to the manufacturer's protocol.

RNA extraction will be performed using RNeasy mini kit (Qiagen), while reverse transcription will be performed using the High-Capacity cDNA Reverse Transcription Kit (Applied Biosystems). Both manufacturers are well established and provide protocols which will be used for the procedures.

Gene expression analysis will be performed using TAQMAN® Gene Expression assays. Each TaqMan Gene Expression assay includes target primers and a sequence-specific probe that is optimized for the best functional performance. Thermo Fisher Scientific's TaqMan Gene Expression Assays are extremely reliable, since all primers are tested and verified by the manufacturer.

Antibodies for pain-related markers will be ordered from known and established vendors (Abcam, Novus Biologicals, Lsbio, Jackson Immunoresearch). Adequate production and quality control of antibodies that will be used is tested and ensured by the manufacturers.

iPS cell lines—The iPS lines were obtained by the iPS core from Coriel Institute for medical Research. Three different lines were reprogramed from dermal fibroblasts isolated from different healthy patients:

TABLE 1

| | Parent | Age at Time | Sampling |
|---|---|---|---|
| iPSC Line | Line | of Collection | Site |
| CS83iCTR-33 | GM02183 | 21 | |
| CS15iCTR-n | GM03815 | Unknown | Skin/Arm |
| CS25iCTR-18n | ND30625 | 76 | |

iPS cell line validation - the cell lines were validated by CS iPS core facility using short tandem repeat analysis (STR).

Rheological characterization of the hydrogel constructs cultured in different conditions will be made using a DHR-2 rheometer (TA Instruments), with a 20 mm diameter parallel plate. Imaging will be performed using the following instruments, software, and methods:

μMRI imaging will be performed with a small animal magnetic resonance imaging scanner—Bruker BioSpec 9.4T (94/20) with Avance III electronics 9.4T.

Carl Zeiss Axio Imager Z1 fluorescent microscope (Carl Zeiss) equipped with ApoTome and AxioCam HRc cameras will be used to image slides post-immunostaining.

High speed Hamamatsu BT-CCD camera with a GFP filter will be used to image nociceptor activity, whereas the data will be analyzed using the open source CalmAn analysis pipeline with custom analysis scripts.

Maestro MEA platform and recording software (Axion Biosystems) will be used to measure nociceptor response to secreted factors. In addition, wave form events will be further validated and sorted into individual neurons using Offline Sorter (Plexon).

### Example 5. INC Delivery, Survival, Differentiation, and Matrix Secretion in PEG-Fibrinogen Microspheres, and Study of iNCs Microspheres to Regenerate IVD in Large Animal Model

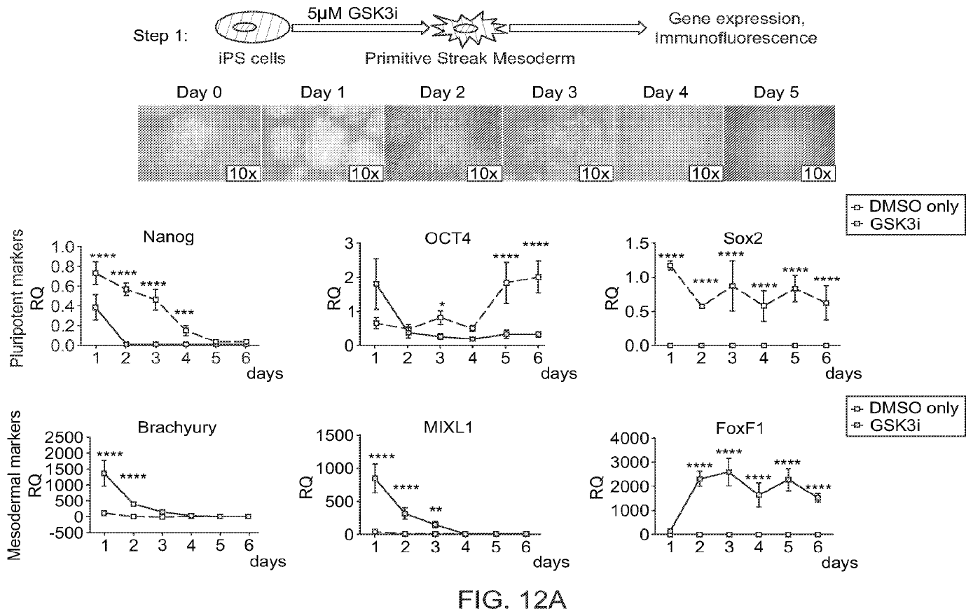

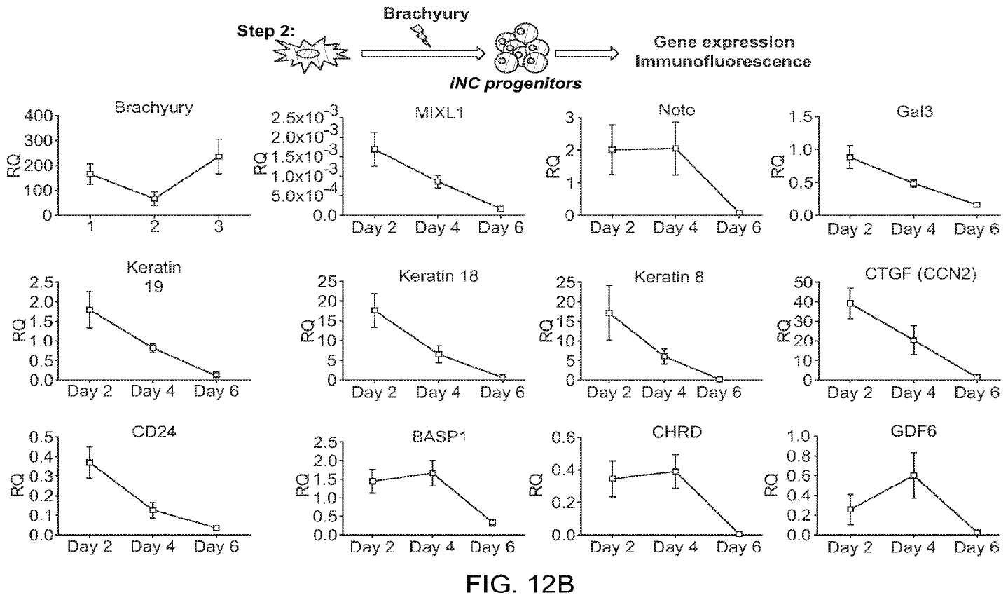

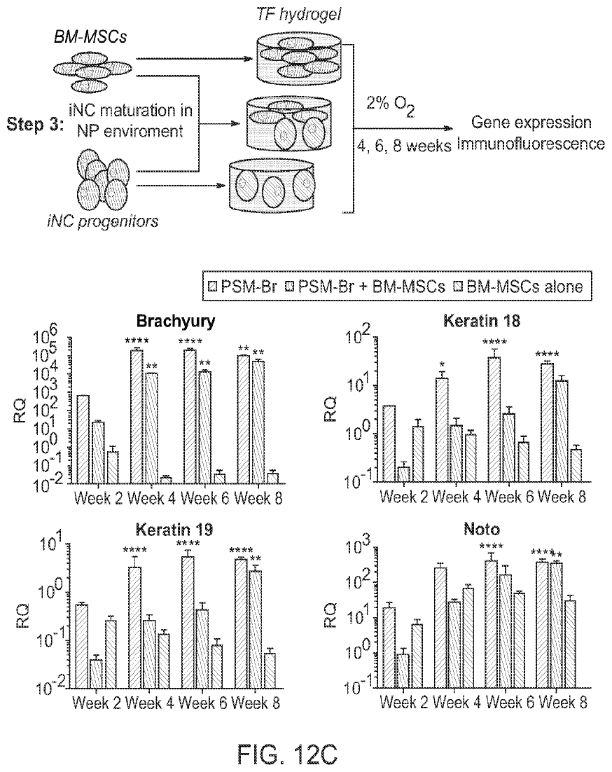

The iNCs will be generated from Luciferase reporter gene-labeled iPSCs using the protocol described in FIGS. 12A and 12B. Healthy IVD explants will be harvested from fresh porcine cadavers with PrimeGrowth™ IVD isolation kit according to manufacturer′ protocol and cultured in PrimeGrowth™ media on an orbital shaker in 37° C. and 5% CO₂. IVD degeneration will be simulated enzymatically. MMP-3 (10 μg/mL) and ADAMTS-4 (10 μg/mL) will be injected into the center of the NP. IVD degeneration is expected to occur within 8 days after induction and will be verified using μMRI. iNC will be either encapsulated in PEG-Fibrinogen microspheres, or suspended in PuraMatrix™ hydrogel and injected to the IVD explants according to Table 1. The microspheres will be crosslinked to provide both biomechanical support and structure to the new forming NP tissue. Cell survival will be evaluated with bioluminescent imaging (BLI) longitudinally and by Live/Dead assay at harvest. The differentiation of the cells and matrix secretion will be assessed with μMRI, gene expression analysis, biochemical assays, immunostaining for aggrecan and collagen type 2. See FIG. 13 for a schematic of the experimental design.

TABLE 2
Experimental group.

| | | Cells | Carrier | Time | Analysis and |
|---|---|---|---|---|---|
| Group | Degeneration | injected | injected | Points | N per group |
| 1. | No | No cells | No injection | Day 0, | μMRI, |
| 2. | degeneration | | Saline | Week 4 | Live/Dead, |
| 3. | (healthy | | PURAMATRIX ™ | (baseline + | IF, Gene |
| 4. | control) | | PEG-F MSs | culture | expression, |
| | | | | control) | biochemical |
| | | | | | assays: |
| | | | | | 4 Ca * 2 TP * 2 |
| | | | | | An * 3 (N) = 48 |
| 5. | | 10⁵iNC- | Saline | Day 0, | BLI, μMRI, |
| 6. | | Luc | PURAMATRIX ™ | Week 4, | Live/Dead, |
| 7. | | | PEG-F MSs | Week 8, | IF, Gene |
| | | | | Week 12 | expression, |
| | | | | | biochemical |
| | | | | | assays: |
| | | | | | 3 Ca * 4 TP * 2 |
| | | | | | An * 3(N) = 72 |
| 8. | MMP-3 and | No cells | No injection | | μMRI, |
| 9. | ADAMTS-4 | | Saline | | Live/Dead, |
| 10. | induced | | PURAMATRIX ™ | | IF, Gene |
| 11. | degeneration⁴⁹ | | PEG-F MSs | | expression, |
| | | | | | biochemical |
| | | | | | assays: |
| | | | | | 4 Ca * 4 TP * 2 |
| | | | | | An * 3(N) = 72 |
| 12. | | 10⁵iNC- | Saline | | BLI, μMRI, |
| 13. | | Luc | PURAMATRIX ™ | | Live/Dead, |
| 14. | | | PEG-F MSs | | IF, Gene |
| 15. | | | Saline | | expression, |
| 16. | | 5 × 10⁵iNC- | PURAMATRIX ™ | | biochemical |
| 17. | | Luc | PEG-F MSs | | assays: |
| | | | | | 2 CT * 3 Ca * 4 |
| | | | | | TP * 2 An * |
| | | | | | 3(N) = 144 |

| | |
|---|---|
| Total IVD organ cultures (12 IVDs can be | 336 IVDs (28 |
| harvested per pig for organ culture) | porcine spines) |

PEG-F MSs—PEG-Fibrinogen microspheres, CA—Carrier, TP—Time points, An—method of analysis, CT—cell types.

Based on our preliminary data we believe that iNCs will survive in PEG-F microspheres injected into the IVD explants. If μMRI monitoring demonstrates naturally occurring serine proteinases cause unintended IVD tissue degeneration in control groups, we can add protease inhibitors after 7 days, such as the Trasylol, to slow down the degradation of the explant. We anticipate that with time PEG-F will be replaced by new matrix, iNCs will fully integrate and will have paracrine effect on porcine NP cells, resulting in a higher expression of NP markers and increased matrix secretion compared to the controls. In some implementations, human bone marrow-derived MSCs are co-injected with iNCs-loaded microspheres, or co-encapsulated with iNCs in microspheres.

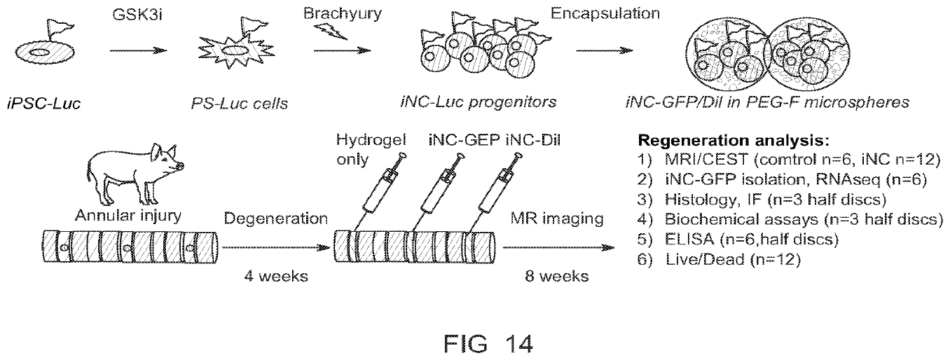

IVD degeneration will be induced using an annular puncture on three non-adjacent spine levels (L1-L6). iNCs will be generated from iPSCs pre-labeled with GFP reporter gene for identification, encapsulated in PEG-Fibrinogen microspheres and injected into the degenerated IVDs 4 weeks post-induction. The regeneration process will be monitored using 3T MRI. Once the IVDs are harvested, one of the discs will be used to sort the GFP+ cells from the NP using FACS and characterized using RNA sequencing. The second treated and the “hydrogel only” disc will be used to 1) evaluate the iNC survival by Live/Dead assay on a small biopsy, 2) assess the regeneration of the disc and matrix composition by DMMB assay and immunofluorescence on histological sections. See FIG. 14 for the experimental design.

Induced disc degeneration was demonstrated to cause rapid reduction in GAG and water content in the NP. Discs treated with the iNC-loaded microspheres are believed to have a significant attenuation in the rate of degeneration, which will be associated with an increase of water content and disc height (outcomes of regeneration) at 8 weeks after treatment. In some implementations, additional minimally invasive injections of cells are conducted. In some implementations, co-injection with human MSCs is also performed besides injection of iNC-loaded microgel.

We set the significance level at 5% (α≤0.05) and sought to choose a sample size that will minimize the probability of Type II error (β to ≤10%). We find that at least 5 IVDs per group are necessary for MRI and BLI studies. N=3 is minimal for gene expression assays, biochemical assays (for each assay it is sufficient to use half of the IVD), histological analysis and IF staining. GraphPad Prism 6 software will be used to analyze the data. Longitudinal data analysis will be conducted using a one-way ANOVA or two-way ANOVA with repeated measures and the Bonferroni post-test. This study looks at the potential of IVD rejuvenation using iNCs and new injectable microspheres.

### Example 6. Study of iNC-Microgel/Microtissue or iNC-Bulk Hydrogel Injection in Attenuating IVD Degeneration in a Pig Model

Mini pigs will undergo lumbar disc puncture at 3 non-consecutive lumbar levels (L1/L2, L3/L4 and L5/L6) using a 14G spinal needle. Successful disc degeneration induction will be confirmed by 3T MRI at week 4 prior to treatments administration. In a second procedure porcine IVDs will be injection with total volume of 100 μl, 10% of which will be contrast agent. The experimental groups will include: (1) human iNC-microgel (2.5×10⁶/ml); (2) human iNC-microgel (2.5×10⁶/ml) with 4 weeks immunosuppression and (3) saline control. All three levels of each pig will be treated with the same treatment to identify systemic and local effects (n=6, total 18 pigs). To evaluate the preliminary safety of the different treatment components (cells and biomaterials), blood samples will be collected at baseline, 4 weeks after IVD degeneration and every 2 weeks after treatment. Inflammation and pain observations will be performed by the vivarium staff regularly. Immune response and presence of infiltrating immune cells will be monitored using ELISA for anti-human IgM antibodies present in the serum, and cell survival will be evaluated using gene expression analysis using porcine and human primers and histologically in the end of the study. Each animal will undergo gross necropsy post-sacrifice. For additional safety evaluation biodistribution assay will be executed using PCR for human genes on DNA isolated from tissues harvested at week 8.

Animal procedures and intradiscal injections: Briefly, healthy female Yucatan miniature pigs with an average age of 10 months and 40-60 kg weight will be used. Female pigs will be used since females have been shown to have the higher predisposition to disc degeneration. Animals will be anesthetized following institutional standard procedures. Anesthesia will be maintained using 1-3.5% inhaled isoflurane for the duration of the procedure. IVD degeneration will be induced by a single annular injury. Specifically, under fluoroscopic guidance, a 14G VertePort needle (Stryker, Kalamazoo, MI) will be used to percutaneously penetrate and injure the annulus fibrosus of the IVD parallel to the endplate via a posterolateral approach. This procedure will be repeated at three non-consecutive lumbar levels (L1/L2, L3/4 and L5/L6). Four weeks later, the animals will be imaged with MRI (Siemens Medical Solutions USA, Inc., PA) to verify degeneration. After the degeneration is verified, cell-laden microgels will be resuspended in 100 μl solution (10% contrast agent) and injected into the center of the degenerated discs using a 18G spinal needle under fluoroscopic guidance. To administer immunosuppression and collect blood, an in-dwelling jugular catheter will be placed during the same procedure. Immunosuppression will be induced with tacrolimus 0.1 mg/kg just prior to cell transplantation. Tacrolimus will be administered IV at a dose of 0.025 mg/kg twice a day for 4 weeks. These doses were shown to be enough in experiments performed at our institution using human neural progenitor cells transplanted into the spinal cord.

MRI in vivo: Imaging experiments will be performed using a 3T clinical MRI scanner (Magnetom Verio; Siemens Healthcare, Erlangen, Germany). Briefly, the animals will be placed in the right decubitus position with body array coils centered on the posterior aspect spinous process. Throughout the imaging procedures, anesthesia will be maintained with isoflurane (1%-3.5%). qCEST MRI, T1, T2 and T1ρ mapping will be performed in the axial plane for each IVD and the scan time for each animal will be approximately 40 min. CEST MRI will be performed using a two-dimensional reduced field of view TSE CEST sequence (TR/TE 1/4 10,500/10 ms, two averages, single shot).95 For each IVD, images will be acquired in the axial plane with 3-mm slice thickness, 140×40-mm² field of view, and 1.1×1.1-mm² spatial resolution. The CEST saturation module consists of 39 Gaussian-shaped pulses, with a duration tp-80 ms for each pulse and an interpulse delay td=80 ms (duty cycle=50%, total saturation duration Ts-6240 ms) at saturation flip angle 900, 1500, 2100, and 3000 B1 amplitudes=flip angle/(gtp)=0.73, 1.22, 1.71, and 2.45 μT; the Z-spectrum will be acquired with 10 different saturation frequencies at ±1.6, ±1.3, ±1.0, ±0.7, and ±0.4 ppm. The B0 field will be corrected using a water saturation shift referencing (WASSR) map. T1 mapping will be performed using an inversion recovery TSE sequence with seven different inversion times (TI=50, 150, 350, 700, 1050, 1400, and 2000 ms), TR/TE-6000/12 ms, FOV=280×280 mm², and spatial resolution=1.1×1.1×3 mm³. T2 mapping will be performed using a TSE sequence with various echo delays (TE=12, 25, 50, 99, 199, and 397 ms; TR=6000 ms), FOV=280×280 mm², and spatial resolution=1.1×1.1×3 mm³.

Anti-human antibody detection in serum: Microtiter wells will be coated with iNC cell lysate. Porcine serum collected at different time points (baseline, 4 weeks post degeneration induction, 2-, 4-, 6- and 8-weeks post treatment) will be added to the antigen-coated wells. A mixture of FITC conjugated anti-IgM (Bio-Rad) and secondary AlexaFluor568 conjugated anti-IgG will be added. The fluorescence will be measured. Purified anti-pig antibodies (Fitzgerald Industries), IgG and IgM, will be used as standards.

Analysis of harvested of IVDs and DRGs: At week 12, IVDs will be harvested from the injected non-consecutive lumbar levels (L1/L2, L3/L4 and L5/L6), using non-degenerated levels (L2/L3 and L4/L5) as control for gene expression (n=3) and histological analyses (n=3).

For gene expression (n=3/group), RNA will be isolated from the NP tissues and TaqMan gene expression assays to analyze the following genes: 1) inflammation-related genes, NFKBAI, TNFα, IL1β, IL6, IL8, IL17, and IFNγ; 2) pain-related genes, CGRP, NGF and BDNF, and 3) IVD degeneration markers CNN2, MMP3, AGC, Col I and II and 4) human notochordal markers. Pigs for histology (n=3/group) will be anesthetized and transcardially perfused with saline and 4% PFA. IVD and DRG tissues will be collected and fixed for an additional two hours in 4% PFA. The DRGs will be transferred to 30% sucrose for 48 h for cryoprotection before sectioning at 35 μm. IVDs will be decalcified and embedded in paraffin, sections will be analyzed for morphological changes using standard H&E, and Picrosirius Red/Alcian Blue stains. To investigate the degeneration and inflammation state of the IVDs staining will be performed against TNFα, IL-1β, IL-6 and NP degeneration markers (CNN2, MMP3). To validate the qCEST results, the IVDs will be stained with pain markers GAP43, TRPV1 NGF, BDNF and CGRP.

Biodistribution: Different organs (brain, bone marrow, liver, lungs, heart muscle, skeletal muscle, and spleen, spinal cord) will be biopsied immediately after the non-perfused animal has been euthanized and will be snap-frozen. Then the tissues will be homogenized, and DNA extracted using a DNA extraction kit (Qiagen). Since the pigs will be treated human iNC that were transfected with human Brachyury gene, the DNA samples will be tested for human Br using quantitative PCR and normalized to the 18S housekeeping gene.

We believe the iNC will survive and retain their phenotype in the porcine IVDs without inducing immune response or rejection, especially since the cells are encapsulated in microgels. In case the survival of cells will be significantly lower in immunocompetent animals comparing to immunosuppressed ones, we will consider using immunosuppression for a short period after the treatment in all subsequent experiments. If needed, short term immunosuppressive drug treatment can be used in the clinical study. Ruminants of the cells are not expected in any tissues outside the IVD. In some implementations, chemical crosslinker is added to the microgels, to increase mechanical strength of the microgels thereby reducing cell leakage from the microgels.

Furthermore to test efficacy of the therapeutic candidates in IVD degeneration, each porcine IVD (3 levels in each animal) will be treated with one of the following candidates: (1) iNC-microgel (2) iNC-microtissue; (3) iNC-bulk hydrogel; (4) microgel only; (5) iNC in saline, and (6) saline control. The 6 treatments will be randomized between the 18 animals, so each treatment or control will be injected into 9 IVDs. The porcine IVDs will be degenerated, treated, and monitored as described above. To study the efficacy of the treatment, pigs will undergo MRI analysis (qCEST, Trho) pre- and post-treatment at week 2, 4 and 8. For data analysis, group effects and, if applicable, temporal effects will be evaluated. To complement the imaging analysis of regeneration, 3 porcine IVDs per group will be used for GAG quantification using DMMB assay normalized to BCA (n=3) and 3 out 9 IVDs will be used for histology and IF analysis (using markers for iNCs, differentiation, matrix degradation, inflammation and pain). To tap into the mechanism of action of the different delivery systems, 3 IVDs will be harvested, enzymatically digested, cells isolated and used for scRNA-seq analysis of cell fate and differentiation state of the injected cell and the degeneration state of the host NPCs.

Single cell RNA-sequencing (scRNA-seq): For each sample the cells will be isolated. Chromium Single Cell 3′ v3 libraries with ˜3,000 cells will be prepared on a Chromium Controller with chips and reagents from Single Cell Gene Expression v3 kits following the manufacturer's protocols (10× Genomics). Then, the libraries will be sequenced using paired-end sequencing (28 bp Read 1 libraries, and 91 bp Read 2) with a single sample index (8 bp) on an Illumina NovaSeq. Samples will be sequenced to a depth of >50,000 raw reads per cell, with raw sequencing data analyzed and visualized with Cell Ranger and Loupe Cell Browser. Only single cell libraries that pass quality control filters will be aggregated across all experimental batches and analyzed together. We will compare their cell subsets and cell types across different samples. We will further compare the expressions of NP, AF or NC-relevant genes at single cell resolution. The analysis will include two phases. First the human cells will be separated from porcine ones. Secondly the human cell phenotype will be compared to iNCs harvested before injection and to phenotypes of human IVD cells. The porcine cells will be profiled based on known markers and their response to degeneration and regeneration will be compared to human degenerative IVD cells.

Since we saw protective effect of iNC in bulk hydrogel, we believe at least the same effect using FF hydrogel and more significant regeneration using microgel or microtissue approach. The idea of microtissue is to allow the cells to attach to the biomaterials, secrete some ECM proteins and form a microstructure that would support NP tissue formation before he injection into a harsh environment of the degenerated IVD. Therefore, we conceive that pre-conditioned microgels or the microtissues will have the better therapeutic outcomes than other groups. In some implementations, we can also increase the cell density or extend the length of the regeneration phase of the experiment to 12 weeks.

To select an iNC cell density in microgel/microtissue, the efficacy of different cell densities will be tested in degenerated discs using iNC-microgel/microtissue (0.5×10⁶/ml, 2×10⁶/ml and 5×10⁶/ml) compared to each other and the saline control. Each of the 3 degenerated IVDs per pig will be treated with microgels different cell density in randomized levels. The porcine IVDs will be degenerated, treated, and monitored. The outcome measures will be MRI in vivo (n=6), histology (n=3) and GAGs (n=3).

NP tissue has low cellularity and low nutrient supply, therefore too many cells will lead to apoptosis and could prevent effective regeneration. On the other hand, too low cell density will not be able to secrete enough matrix and restore the tissue structure. Since the space in the IVD is limited, so will the doses/volume of microgels be, density of loaded iNCs can be adjusted.

To test the reproducibility of a therapeutic candidate at a selected cell density, the therapeutic candidate will be tested against untreated control. IVDs of 3 levels in 6 pigs will be degenerated. One randomized level in each pig will be treated with the optimized iNC therapy (n=6), one level will be treated with pNPC injected in saline (n=6), and one level will be left untreated serving as internal control (n=6). The IVDs will be degenerated, treated, and the efficacy of the treatment will be monitored.

Primary nucleus pulposus-derived cells (NPCs) are the closes available to cell therapy, however the supply of human NPCs is extremely limited. In case of the porcine model, porcine NPCs allogenic can be easily obtained from other pigs thus being attractive “gold standard” control to iNCs. We believe the therapeutic candidate will show reproducibility and lead to comparable results to the ones achieved other studies or with NPCs.

We set the significance level at 5% (α≤0.05) and sought to choose a sample size that will minimize the probability of Type II error (β to ≤10%). Based on power analysis of published and preliminary data we find that at least n=3 is needed for in vitro assays, n=6 IVDs per group are necessary for porcine MRI studies, n=3 for scRNA-seq, and n=3 for minimal histology and staining. GraphPad Prism 9 will be used for analysis. Longitudinal data analysis will be conducted using a one-way ANOVA or two-way ANOVA with repeated measures and the Bonferroni post-test.

### Example 7. Attenuating Discogenic Low Back Pain with Injectable Stem Cell-Loaded Thermo-Responsive Microgels in a Rat IVD Degeneration Model

Chronic low back pain (LBP) affects over half a billion people worldwide and is commonly associated with intervertebral disc (IVD) degeneration. IVD regeneration remains an unmet clinical need, as current therapies such as surgical intervention and pain alleviation do not address the disease at its root cause—deterioration of the nucleus pulposus (NP) in inner core of the IVD. Notochordal cells (NCs) give rise to NP cells in development, have the potential to replenish NP cells and regenerate the IVD. NC are scarcely available in the adult but could be differentiated from induced pluripotent stem cells (iPSCs). Our strategy is to develop an injectable cell therapy for LBP leveraging iPSC-derived NCs (iNCs) in combination with a cell delivery system based on hydrogel microparticles (microgels). The thermo-responsive hydrogels allow to encapsulate and precondition cells without introducing any cytotoxic crosslinker. The potency of this cell therapy was examined in vivo in a rat IVD degeneration model.

The iPSCs were differentiated into iNCs using a three-step process via presomitic mesoderm and overexpression of Brachyury transcription factor (Sheyn et al., Theranostics, 2019, 9 (25): 7506-7524 and U.S. Patent Application Publication No. US2020/0093961). The iNCs were encapsulated in fibrinogen-based thermo-responsive hydrogels in a microfluidic device at 10 million cells/ml gel. The microgels were purified to aqueous solutions and cultured in media in hypoxic conditions (2% 02, 5% CO₂) for 7d (preconditioning). For the in vivo experiment, fluoroscopy-guided percutaneous needle injury was performed in rat lumbar discs (L4-5 & L5-6). After 2 weeks, iNC-loaded microgels were injected into the degenerated discs. The IVD height were evaluated using μCT and the discogenic pain was evaluated using biobehavioral tests.

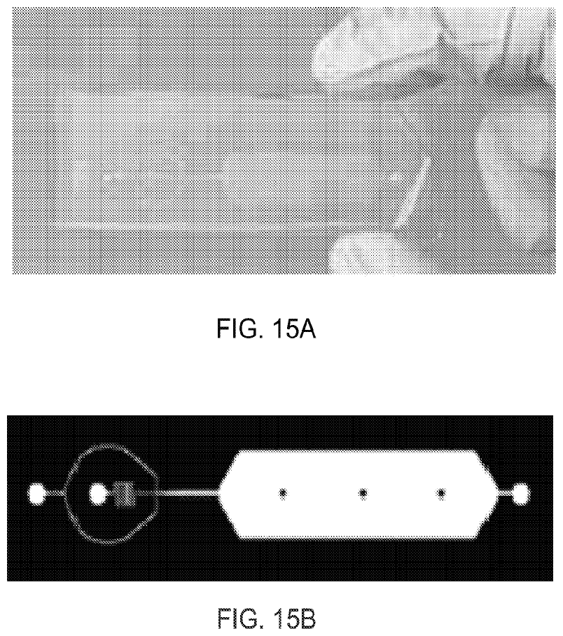

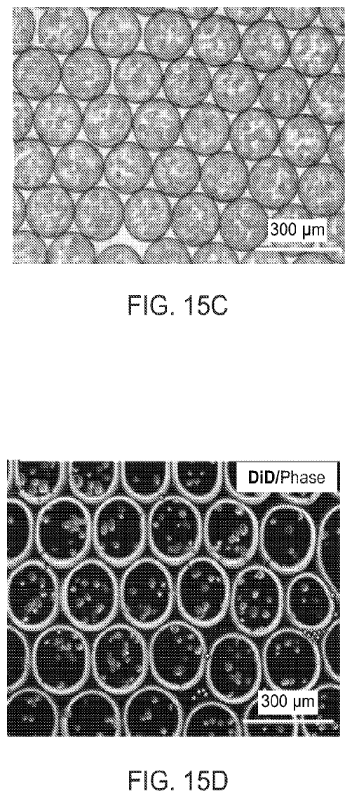

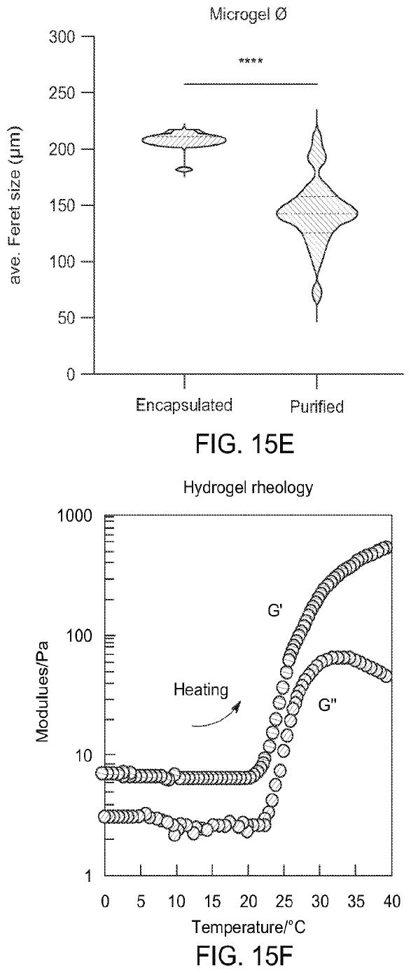

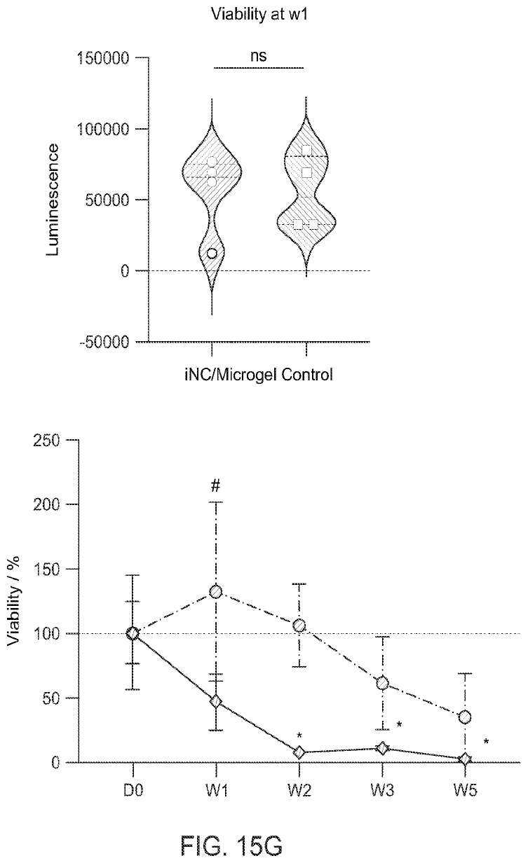

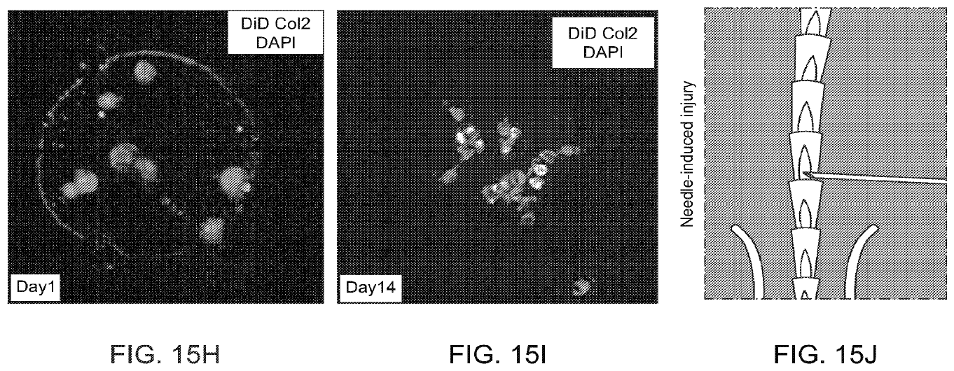

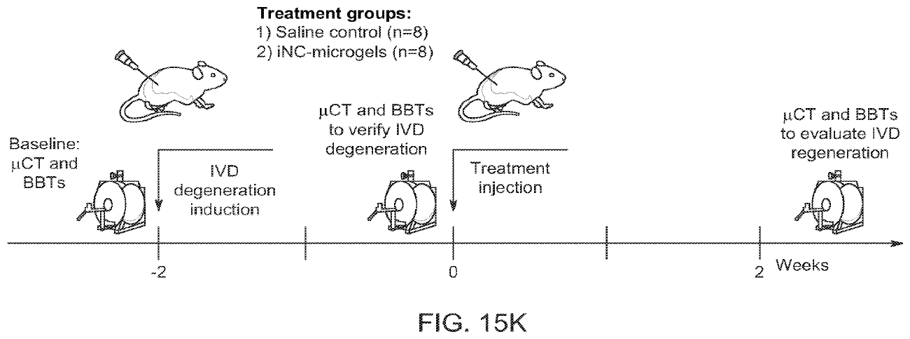

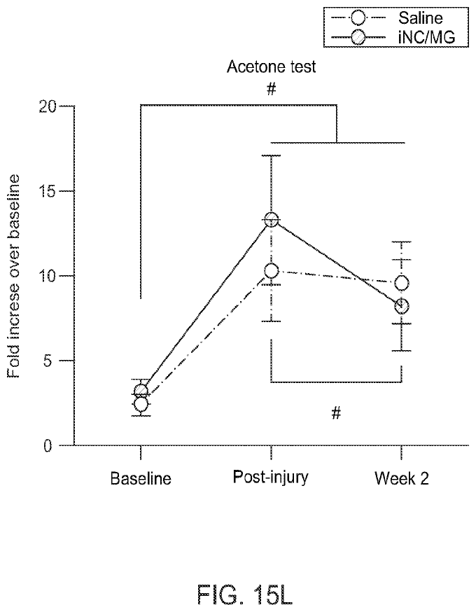

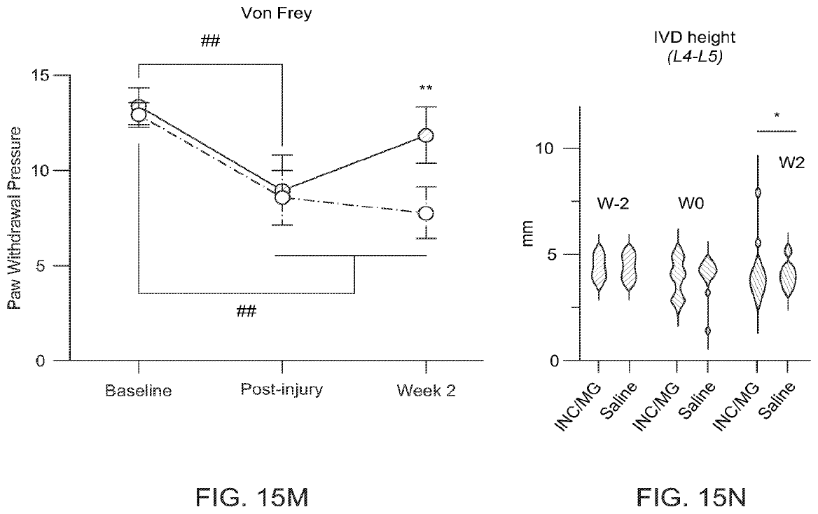

Microfluidic device was designed and fabricated (FIG. 15A, 15B). The microgels have unified morphology and consistent cell density (FIG. 15C, 15D). The diameter of the microgels was ˜150 μm in average after the purification (FIG. 15E). Thermal gelation occurs around 21° C. (FIG. 15F). The human cells encapsulated in microgel maintained comparable viability to the bulk hydrogel control (FIG. 15G). The 14d preconditioning of iNC-loaded microgels promoted ECM (Col2) deposition (FIG. 15H, 15I). In our needle-induced IVD degeneration rat model (FIG. 15J, 15K), the intradiscal injection of iNC-loaded microgels resulted in statistically higher cold hypersensitivity detected with acetone test (FIG. 15L) and paw withdrawal pressure of rats compared to saline control (FIG. 15M), indicating less mechanical allodynia 2 weeks post treatment. The iNC-loaded group also showed increased IVD height at L4-L5 than saline control.

The robust thermos-responsive property of the fibrinogen-based hydrogel permitted reliable, high-quality microencapsulation of cells without the need for cytotoxic UV crosslinking. The presence of fibrinogen resulted in a friendly environment for cells to attach, survive, and secret ECM before injection. The preconditioned iNC-loaded microgels showed positive effects in alleviating pain and regenerating IVDs as early as 2 weeks post-surgery.

Thermo-responsive microgels allowed for the preconditioning and effective injection of stem cells in vivo. The high-quality injectable microgels and regenerative iNCs provides a new avenue for the clinical translation of minimally invasive cell therapies treating discogenic low back pain.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

The invention claimed is:

1. An injectable composition, comprising a dispersion comprising microgel particles and human induced pluripotent stem cell (iPSC)-derived notochordal cells (iNCs), wherein the iNCs are encapsulated in the microgel particles, and the size of the microgel particles is between 30 μm and 1000 μm.

2. The injectable composition of claim 1, wherein the iNCs secrete collagen type II, and the microgel particles encapsulating the iNCs are deposited with the collagen type II.

3. The injectable composition of claim 1, wherein the microgel particles each comprises a cross-linked polymeric network comprising:
a plurality of first polymeric segments derived from a polyoxyalkylene, and
a plurality of second polymeric segments derived from a bioadhesive polypeptide or polysaccharide,
wherein the first polymeric segments and the second polymeric segments are bonded together to form a polymeric network.

4. The injection composition of claim 3, wherein the polymeric network comprises linking groups connecting the first polymeric segments to the second polymeric segments, optionally the linking groups comprising an ester group or being derived from an acrylate.

5. The injectable composition of claim 3, wherein the bioadhesive polypeptide or polysaccharide comprises fibrinogen, laminin, or hyaluronic acid.

6. The injectable composition of claim 5, wherein the bioadhesive polypeptide or polysaccharide presents or is coupled with a thiol group or a methacrylate group, and the polyoxyalkylene is coupled with an acrylate group.

7. The injectable composition of claim 6, wherein the bioadhesive polypeptide or polysaccharide comprises fibrinogen, and the polyoxyalkylene comprises polyethylene glycol.

8. The injectable composition of claim 5, wherein the polyoxyalkylene comprises at least one block derived from propylene oxide monomers and at least one block derived from ethylene oxide monomers.

9. The injectable composition of claim 8, wherein the polyoxyalkylene is an ABA triblock copolymer, wherein the A blocks are derived from the ethylene oxide monomers and the B block is derived from the propylene oxide monomers.

10. The injectable composition of claim 1, wherein the iNCs are prepared by a process comprising: culturing human iPSCs in the presence of a glycogen synthase kinase 3 (GSK3) inhibitor (GSK3i) to form primitive streak (PS) cells; transfecting the PS cells with a vector encoding Brachyury to overexpress Brachyury; expressing Brachyury in the PS cells, wherein expression of Brachyury by the vector encoding Brachyury in the PS cells induces formation of human iNCs, and the human iNCs express Brachyury, Keratin 18, and Keratin 19.

11. The injectable composition of claim 1, wherein the microgel particles are between 50 μm and 250 μm in size, and the iNCs are encapsulated in the microgel particles at a number ratio of iNC-to-microgel particle being between 1:1 and 80:1.

12. The injectable composition of claim 1, in a nucleus pulposus (NP)-specific medium for culturing in a hypoxic condition for a period of time selected for the iNCs to secrete an extracellular matrix protein comprising collagen type II.

13. A method for treating a subject with intervertebral disc degeneration and/or discogenic low back pain, and/or modulating the intervertebral disc degeneration in the subject, the method comprising injecting an effective amount of the injectable composition of claim 1 into a nucleus pulposus, a vertebral disc, an invertebral disc, or clefts of a nucleus pulposus of an intervertebral disc of the subject.

14. The method of claim 13, wherein the injectable composition is intradiscally injected to the nucleus pulposus of the subject.

15. The method of claim 13, wherein at least 1×10⁶, 2×10⁶, or 3×10⁶ human iNCs are administered to the subject, and wherein the microgel particles each comprises a cross-linked polymeric network comprising a plurality of poloxamer segments and a plurality of fibrinogen segments, wherein the poloxamer segments and the fibrinogen segments are bonded together via linking groups to form the polymeric network.

16. The method of claim 13, wherein treating the subject and/or modulating the intervertebral disc degeneration comprises an increase in disc height and/or an increase in cold hypersensitivity of the subject.

17. A method for preparing the injectable composition of claim 1, comprising:
mixing an aqueous solution comprising a precursor polymer to forming the microgel particles with the iNCs to form a precursor-cell mixture;
subjecting the precursor-cell mixture to microinjection or micronization into an oil phase, wherein the precursor-cell mixture is microinjected or micronized to form a dispersion of microparticles in the oil phase; curing the microparticles in response to a stimulus selected for inducing gelation of the microparticles and purifying the microparticles to remove residue from the oil phase, thereby forming a dispersion of microgel particles which encapsulate the iNCs; wherein the precursor polymer comprises a first polymeric segment derived from polyoxyalkylene and a second polymeric segment derived from a bioadhesive polypeptide or polysaccharide, wherein the first polymeric segment and the second polymeric segment are bonded together, and wherein optionally the stimulus comprises an increase in temperature or an exposure to ultraviolet or visible light.

18. The method of claim 17, further comprising culturing the microgel particles which encapsulate the iNCs in a hypoxic condition for a period of time selected for inducing secretion of an extracellular matrix protein comprising collagen type II by the iNCs and/or for maintaining of at least 50% activity of the iNCs in the microgel particles compared to before encapsulation.

19. The method of claim 17, wherein the first polymeric segment derived from polyoxyalkylene comprises an ABA triblock copolymer, wherein the A blocks are derived from ethylene oxide monomers and the B blocks are derived from propylene oxide monomers, such that the aqueous solution viscosifies in response to the stimulus comprising the increase in temperature, and the microparticles formed from the precursor-cell mixture is thermal-cured to form the dispersion of microgel particles.

20. The method of claim 17, wherein the first polymeric segment and/or the second polymeric segment is modified with a photo-reactive chemical group, such that the aqueous precursor solution becomes reactive in response to the stimulus comprising the exposure to ultraviolet or visible light, and the microparticles formed from the precursor-cell mixture is photo-cured to form the dispersion of microgel particles.
